(19) United States
(12) Patent Application Publication
(10) Pub. No.: US 2024/0050481 A1
(43) Pub. Date: Feb. 15, 2024

(54) COMPOSITION FOR USE IN TREATING DYSTROPHIC EPIDERMOLYSIS BULLOSA

(71) Applicants: OSAKA UNIVERSITY, Osaka (JP); STEMRIM INC., Osaka (JP)

(72) Inventors: Katsuto TAMAI, Suita-shi, Osaka (JP); Yasushi KIKUCHI, Suita-shi, Osaka (JP); Tomoki TAMAKOSHI, Suita-shi, Osaka (JP); Takehiko YAMAZAKI, Toyonaka-shi, Osaka (JP)

(73) Assignees: OSAKA UNIVERSITY, Osaka (JP); STEMRIM INC., Osaka (JP)

(21) Appl. No.: 18/017,149

(22) PCT Filed: Jul. 21, 2021
(86) PCT No.: PCT/JP2021/027279

§ 371 Date: Sep. 29, 2023

(30) Foreign Application Priority Data
Jul. 22, 2020 (JP) 2020-125620

(51) Int. Cl. A61K 35/28; C12N 5/0775; C07K 14/78; A61P 17/00

(52) U.S. Cl. CPC A61K 35/28; C12N 5/0668; C07K 14/78; A61P 17/00

(57) ABSTRACT

The present disclosure includes a composition for use in the treatment of dystrophic epidermolysis bullosa, comprising a blister-derived cell of a patient with dystrophic epidermolysis bullosa that is genetically modified to produce type VII collagen.

### TECHNICAL FIELD

The present application claims priority with respect to Japanese Patent Application No. 2020-125620, which is incorporated herein by reference in its entirety.

The present disclosure relates to compositions for use in treating dystrophic epidermolysis bullosa.

### BACKGROUND

Epidermolysis bullosa is a disease in which adhesive structural molecules responsible for adhesion of the skin tissue are lost or disappeared, and then the epidermis peels off from the dermis and blisters or skin ulcers occur when force is applied to the skin. The disease includes simple epidermolysis bullosa, in which the epidermis is torn to form blisters, junctional epidermolysis bullosa, in which the epidermis is peeled from the basement membrane to form blisters, and dystrophic epidermolysis bullosa, in which the basement membrane is peeled from the dermis.

Dystrophic epidermolysis bullosa is the most common type of epidermolysis bullosa, accounting for about 50% of all epidermolysis bullosa. It is a hereditary disease caused by a mutation in the COL7A1 gene, which encodes type VII collagen. In the structure of the skin, the epidermal basal cells at the bottom of the epidermis are bound to a sheet-like structure called the basement membrane. Type VII collagen forms fibers called anchoring fibrils in the dermis and connects the basement membrane and the dermis. Therefore, if there is an abnormality in the type VII collagen gene, the adhesive function between the basement membrane and the dermis is impaired, resulting in dystrophic epidermolysis bullosa, in which blisters form between the basement membrane and the dermis. Among dystrophic epidermolysis bullosa, severe recessive dystrophic epidermolysis bullosa is a very serious hereditary bullous skin disease that has continued burn-like skin symptoms throughout the body immediately after birth, and cutaneous spinous cell carcinoma (scar cancer) occurs frequently from around 30 years old and leads to death.

There is currently no effective treatment for epidermolysis bullosa, and the development of gene therapy that radically suppresses blistering is required. As such gene therapy, a therapeutic technique is disclosed in which skin cells of a patient are collected, genetically engineered to produce type VII collagen, cultured to form a skin sheet, and transplanted to the patient (Patent Document 1). Also, it has been proposed to subject mesenchymal stem cells lacking the type VII collagen activity to genome editing, differentiate the mesenchymal stem cells capable of producing type VII collagen thus obtained into keratinocytes or fibroblasts, culture the cells to form a skin sheet, and use the skin sheet for treating a patient (see Patent Document 2).

### CITATION LIST

### Patent Documents

Patent Document 1: WO2017/120147
Patent Document 2: WO2018/154413

### SUMMARY OF INVENTION

### Problem to be Solved

Manufacturing the skin sheet requires advanced process control and culture technology, and then involves high-difficulty and high-cost. Therapeutic agents that are easier to manufacture are required.

### Solution to Problem

In one aspect, the present disclosure relates to a composition for use in the treatment of dystrophic epidermolysis bullosa, comprising a blister-derived cell of a patient with dystrophic epidermolysis bullosa that is genetically modified to produce type VII collagen.

### Effect of Invention

The present disclosure provides compositions for use in treating dystrophic epidermolysis bullosa.

### BRIEF DESCRIPTION OF DRAWINGS

FIG. 1 is photographs showing the appearance of blister-derived cells up to 20 days after initiation of culture.

FIG. 2 shows the results of FACS analysis of blister-derived cells and human bone marrow-derived mesenchymal stem cells.

FIG. 3 shows blister-derived cells and human bone marrow-derived mesenchymal stem cells cultured under the condition that induces differentiation into osteoblasts, adipocytes, or chondrocytes. The results of Alkaline phosphatase (ALP) staining, Oil Red O staining O, and Alcian Blue staining are shown.

FIG. 4 shows blister-derived cells and human bone marrow-derived mesenchymal stem cells cultured under the condition that induces differentiation into osteoblasts, adipocytes, or chondrocytes, wherein the blister-derived cells are derived from a patient with dystrophic epidermolysis bullosa different from the patient of FIG. 3. The results of ALP staining, Oil Red O staining O, and Alcian Blue staining are shown.

FIG. 5 shows the amount of type VII collagen expressed or secreted by various cells. FIG. 5 (top) is a photograph showing the result of Western blotting of cell lysate using an anti-type VII collagen antibody (left), and a graph showing quantification of the densities of the obtained bands (right). FIG. 5 (bottom) is a photograph showing the result of Western blotting of the medium in which the cells were cultured (left), and a graph showing quantification of the densities of the obtained bands (right). The “KC” indicates human epidermal keratinocytes. The “FB” indicates human dermal fibroblasts. The “MSC” indicates human bone marrow-derived mesenchymal stem cells. The “BFC” indicates blister-derived cells.

FIG. 6 shows the cleavage of genomic DNA by designed sgRNAs (sgAAVS1-#1 to #3) and their cleavage efficiency.

FIG. 7 is an explanatory diagram of genome editing in which a COL7A1 gene is introduced into the AAVS1 region. HA-R and HA-L indicate portions having homologous sequences, SA indicates a splice acceptor sequence, T2A indicates a T2A sequence encoding a T2A peptide, Puro indicates a puromycin resistance gene, and CAG indicates a CAG promoter sequence. The length from F2 to R2 in the wild-type genome (top) is 1952 bp, and the length from F1 to R1 and that from F2 to R2 in the genome into which the COL7A1 gene was introduced (bottom) is 1246 bp and 14249 bp, respectively.

FIG. 8 shows the amount of type VII collagen expressed or secreted by various cells after the COL7A1 gene was introduced by CRISPR-Cas9. FIG. 8 (top) is a photograph showing the result of Western blotting of cell lysate using an anti-type VII collagen antibody (left), and a graph showing quantification of the densities of the obtained bands (right). FIG. 8 (bottom) is a photograph showing the result of Western blotting of the medium in which the cells were cultured (left), and a graph showing quantification of the densities of the obtained bands (right). The “FB” indicates human dermal fibroblasts. The “MSC” indicates human bone marrow-derived mesenchymal stem cells. The “BFC” indicates blister-derived cells.

FIG. 9 is an explanatory diagram of production of epidermolysis bullosa model mice. The photograph on the right shows the formed blister.

FIG. 10 shows sectional images of the skin of an epidermolysis bullosa model mouse to which blister-derived cells were injected by intrablister injection. The photograph on the left shows the results of immunostaining for type VII collagen, and the photograph on the right is a merged image of DAPI staining and immunostaining for type VII collagen. The “Control” shows the results of a mouse to which non-genetically modified blister-derived cells were injected, and the “CAG-hCOL7” shows the results of a mouse to which COL7A1 gene-introduced blister-derived cells were injected.

FIG. 11 is sectional images of the skin of an epidermolysis bullosa model mouse into which human bone marrow-derived mesenchymal stem cells or blister-derived cells were injected by intradermal or intrablister injection. The photographs show the deposited area of type VII collagen. From the top, shown are the results of a mouse into which non-genetically modified human bone marrow-derived mesenchymal stem cells were injected by intradermal injection (hMSC intradermal), a mouse into which COL7A1 gene-introduced human bone marrow-derived mesenchymal stem cells were injected by intradermal injection (COL7-hMSC intradermal), a mouse into which non-genetically modified human bone marrow-derived mesenchymal stem cells were injected by intrablister injection (hMSC intrablister), a mouse into which COL7A1 gene-introduced human bone marrow-derived mesenchymal stem cells were injected by intrablister injection (COL7-hMSC intrablister), and a mouse into which COL7A1 gene-introduced blister-derived cells were injected by intrablister injection (COL7-BF intrablister).

FIG. 12 (left) is a schematic diagram explaining an experiment for investigating how much blister-derived cells administered into a blister graft in the blister. FIG. 12 (right) is a graph showing the level of firefly luciferase activity in the blister fluid collected from the blister 30 minutes after administration of the cells into the blister.

FIG. 13 (left) is a schematic diagram explaining an experiment for observing the long-term grafting potential of cells administered to a living body. FIG. 13 (right) shows photographs showing the levels of firefly luciferase activity measured 1 day and 9 months after administration of the cells into the blister. The “MSC” indicates bone marrow-derived mesenchymal stem cells, and the “BFC” indicates blister-derived cells.

FIG. 14 (left) is a graph showing the time-dependent change of firefly luciferase activity level after administration of the cells into a blister. FIG. 14 (right) is a graph showing the level of firefly luciferase activity one month after administration of the cells into the blister.

FIG. 15 shows fluorescence micrographs 72 hours after infection of a GFP lentiviral vector to various cells. The “GFP” means a photograph showing green fluorescence of cells. The “merge” means a superimposition of the bright field photograph on the “GFP” photograph. For “MOI 1”, cells were infected with the lentiviral vector at MOT 1. For “MOI 5”, cells were infected with the lentiviral vectors at MOT 5. The “BFC” is a photograph of blister-derived cells, the “MSC” is a photograph of human bone marrow-derived mesenchymal stem cells, and the “NHDF” is a photograph of normal human adult dermal fibroblasts.

FIG. 16 is a graph showing the GFP positive cell ratio 72 hours after infection with a GFP lentiviral vector at MOT 1 for each cell type.

FIG. 17 is a plasmid map. FIG. 17 (left) shows the structure of the plasmid pLVSIN-EF1α-COL7A1 constructed by the present inventors. FIG. 17 (right) shows the structure of the plasmid pLVSIN-PGK-COL7A1 constructed by the present inventors. The pLVSIN vector is a SIN (self-inactivating) lentiviral vector plasmid, and the vector expresses a human type VII collagen gene under the control of an EF1α promoter (left) or a PGK promoter (right).

FIG. 18 is a schematic diagram showing the gene structure of the lentiviral vector produced by the present inventors. The LVSIN-EF1α-COL7A1 vector shown in FIG. 18 (upper) has an EF1α promoter and a COL7A1 gene in an expression cassette. The LVSIN-PGK-COL7A1 vector shown in FIG. 18 (bottom) has a PGK promoter and a COL7A1 gene in an expression cassette. In addition to HIV-1 LTR (5′LTR and 3′LTR/ΔU3) and the packaging signal (ψ), these vectors contain WPRE (woodchuck hepatitis virus post-transcriptional regulatory element), cPPT/CTS (central polypurine sequence/central termination sequence), and RRE (Rev response element) to improve transgene expression and viral titer.

FIG. 19 is a photograph of blister-derived cells immunostained with an anti-type VII collagen antibody 14 days after infection with the EF1α-COL7A1 lentiviral vector. The “DAPI” is a photograph of DAPI staining. The “EF1α-C7” is a photograph showing the result of immunostaining for type VII collagen. For “mock”, cells were not infected with any lentiviral vector. For “MOI 0.5”, “MOI 1” and “MOI 2”, cells were infected with the lentiviral vector at MOT 0.5, MOT 1 and MOI 2, respectively.

FIG. 20 is a photograph of blister-derived cells immunostained with an anti-type VII collagen antibody 14 days after infection with the PGK-COL7A1 lentiviral vector. The “DAPI” is a photograph of DAPI staining. The “PGK-C7” is a photograph showing the result of immunostaining for type VII collagen. For “mock”, cells were not infected with any lentiviral vector. For “MOI 0.5”, “MOI 1” and “MOI 2”, cells were infected with the lentiviral vector at MOT 0.5, MOT 1 and MOT 2, respectively.

FIG. 21 shows the results of FACS analysis with an anti-type VII collagen antibody 14 days after infection of blister-derived cells with a lentiviral vector having a type VII collagen gene. For the “EF1α-C7”, blister-derived cells were infected with the lentiviral vector containing an EF1α promoter and a COL7A1 gene in the expression cassette. For “PGK-C7”, blister-derived cells were infected with the lentiviral vector containing a PGK promoter and a COL7A1 gene in the expression cassette. For “mock”, cells were not infected with any lentiviral vector. For “MOI 0.5”, “MOI 1”, and “MOI 2”, cells were infected with the lentiviral vector at MOI 0.5, MOT 1, and MOT 2, respectively.

FIG. 22 is a graph showing quantification of the FACS data of FIG. 21. FIG. 22 (left) is a bar graph showing the percentage of type VII collagen-positive cells. FIG. 22 (right) is a bar graph showing the mean fluorescence intensity of type VII collagen-positive cells.

FIG. 23 is a graph showing the time-dependent change of vector copy number in the genome of blister-derived cells infected with a lentiviral vector having a type VII collagen gene.

FIG. 24 shows sectional images of the skin of an epidermolysis bullosa model mouse into which blister-derived cells were injected by intrablister injection, the cells into which a COL7A1 gene was introduced with a lentiviral vector. The “DAPI” is a photograph of DAPI staining. The “C7” is a photograph showing the result of immunostaining for type VII collagen. The “merge” is a merged image of DAPI staining and immunostaining for type VII collagen. The “Uninfected BFC” shows the result of injecting blister-derived cells which were not infected with the lentiviral vector into the mouse by intrablister injection. The “LVSIN-EF1a-C7-infected BFC” shows the result of injecting blister-derived cells to which a COL7A1 gene was introduced with the lentiviral vector into the mouse by intrablister injection.

### DESCRIPTION OF EMBODIMENTS

Unless otherwise specified, the terms used in the present disclosure have meanings generally understood by those skilled in the art in the fields such as organic chemistry, medical science, pharmaceutical science, molecular biology, and microbiology. Definitions of some terms used in the present disclosure are provided below, and these definitions supersede the general understandings in the present disclosure.

Dystrophic epidermolysis bullosa (DEB) is a hereditary disease caused by a mutation in the COL7A1 gene, which encodes type VII collagen, and is known to be characterized in that no type VII collagen is produced or type VII collagen with reduced function due to the mutation is produced. The type VII collagen forms fibers called anchoring fibrils in the dermis and connects the basement membrane and the dermis. The type VII collagen contains a first non-collagen region, a collagen region, and a second non-collagen region from the N-terminus, and forms a triple chain at the collagen region, which is characterized by a repeating sequence of glycine-X-Y. Two molecules bind to each other at the C-terminus and the N-terminus binds to the basement membrane. Examples of mutations include a mutation in which glycine in the collagen region is replaced by a different amino acid, a stop codon mutation that stops protein translation, and a splice site mutation. The mutation may be in one of the alleles or in both. Dystrophic epidermolysis bullosa includes dominant dystrophic epidermolysis bullosa and recessive dystrophic epidermolysis bullosa, and the recessive dystrophic epidermolysis bullosa include severe generalized recessive dystrophic epidermolysis bullosa and other generalized types with relatively mild symptoms. The dystrophic epidermolysis bullosa herein may be any type of dystrophic epidermolysis bullosa, and the causal mutation in the COL7A1 gene may be any mutation.

A blister is an accumulation of fluid such as body fluid or tissue fluid under the epidermis. A preferable blister is a blister in which fluid is accumulated in the space formed between the epidermis and the dermis due to detachment of the epidermis from the dermis. More preferable blister is a blister in which fluid is accumulated in the space formed between the basement membrane of the epidermis and the dermis due to detachment of the basement membrane from the dermis.

In the present disclosure, a blister-derived cell of a patient with dystrophic epidermolysis bullosa refers to an adherent cell collected from a blister of a patient with dystrophic epidermolysis bullosa, and herein also referred to as “a DEB patient blister-derived cell” or “a blister-derived cell”. The cell can be obtained by culturing a blister content of a patient with dystrophic epidermolysis bullosa on a solid phase. In one embodiment, the blister content is a fluid that has accumulated within the blister, which is herein referred to as “a blister fluid”. The blister content can be collected from a blister of a patient with dystrophic epidermolysis bullosa with a tool such as a syringe. For example, when a injection needle of a syringe is pierced into a blister so that the tip of the injection needle is positioned in the space formed between the epidermis and the dermis and the plunger of the syringe is pulled, a blister fluid can be aspirated into the syringe. In one embodiment, a blister-derived cell can be obtained by seeding a blister content in a medium without any enzymatic treatment such as collagenase or dispase treatment and culturing on a solid phase. Specifically, when a blister fluid collected from a blister is directly seeded in a medium and the medium is incubated on a solid phase for a certain period of time, a cell adhering to the solid phase can be used as a blister-derived cell. In this case, it is preferable to obtain cells that have formed a colony on the solid phase. The blister fluid is preferably seeded in the medium within 3 hours, more preferably within 2 hours, and even more preferably within 1 hour after collection. In the present disclosure, a solid phase means a solid support to which a cell can adhere, and includes, for example, plastic or glass culture vessels such as culture dishes, flasks and multiwell plates. In one embodiment, the solid phase is a plastic culture vessel. The solid phase may be coated, and examples of substances for coating include collagen I, laminin, vitronectin, fibronectin, poly-L-lysine, and poly-L-ornithine. In one embodiment, the solid phase is coated with collagen I. The culturing can be performed in a general incubator under the condition such as “37° C., 5% CO₂” or “37° C., 5% O₂, 5% CO₂”. The culture medium may be any medium that can be used for culturing animal cells, and can be, for example, MEM, MEMα, DMEM, GMEM, RPMI 1640, MesenCult™ (STEMCELL Technologies), Mesenchymal Stem Cell Growth Medium 2 (PromoCell), MSCGM Mesenchymal Stem Cell Growth Medium (Lonza), Cellartis MSC Xeno-Free Culture Medium (Takara Bio), or a mixture thereof. Among them, a medium for culturing mesenchymal stem cells such as MesenCult™, Mesenchymal Stem Cell Growth Medium 2, MSCGM Mesenchymal Stem Cell Growth Medium, Cellartis MSC Xeno-Free Culture Medium is preferably used. The medium is preferably a serum-free medium. The culture period may be any period sufficient for the cell to adhere to the solid phase, and can be e.g., 1 day to several months (for example, for 2, 3 or 4 months), 1 day to 1 month, 1 day to several weeks (for example, 2, 3 or 4 weeks), 1 days to a week.

In an embodiment, the DEB patient blister-derived cell has one or more characteristics selected from:
1) adherent to a solid phase;
2) positive for one or more surface markers selected from the group consisting of CD73, CD105 and CD90;
3) negative for one or more surface markers selected from the group consisting of CD45, CD34, CD11b, CD79A, HLA-DR and CD31;
4) incapable of differentiating into an osteoblast, or less capable of differentiating into an osteoblast than a bone marrow-derived mesenchymal stem cell;
5) incapable of differentiating into an adipocyte, or less capable of differentiating into an adipocyte than a bone marrow-derived mesenchymal stem cell; and
6) incapable of differentiating into a chondrocyte, or less capable of differentiating into a chondrocyte than a bone marrow-derived mesenchymal stem cell.

In the present disclosure, the expression that a cell is “incapable of differentiating” into an osteoblast, an adipocyte or a chondrocyte means that differentiation of the cell into an osteoblast, an adipocyte or a chondrocyte cannot be detected by a standard detection method (such as staining) after induction of differentiation with a standard condition.

In an embodiment, the DEB patient blister-derived cell is a CD73-positive, CD105-positive, and CD90-positive cell. In another embodiment, the DEB patient blister-derived cell is a CD45-negative, CD34-negative, CD11b-negative, CD79A-negative, HLA-DR-negative, and CD31-negative cell. In a further embodiment, the DEB patient blister-derived cell is a cell that is less capable of differentiating into an osteoblast, an adipocyte and a chondrocyte than a bone marrow-derived mesenchymal stem cell. In a further embodiment, the DEB patient blister-derived cell is a cell that is less capable of differentiating into an osteoblast and an adipocyte than a bone marrow-derived mesenchymal stem cell, and incapable of differentiating into a chondrocyte.

In the present disclosure, a blister-derived cell of a patient with dystrophic epidermolysis bullosa that is genetically modified to produce type VII collagen is used. As used herein, the term “cell that is genetically modified to produce type VII collagen” means a cell that is genetically modified to produce a functional type VII collagen (ie, a type VII collagen capable of forming anchoring fibrils).

In the present disclosure, genetic modification of a cell means both modification of a gene in the genome of the cell and modification of the cell to express a gene from a nucleic acid construct outside the genome (such as a vector). That is, the expression “genetically modifying a cell to produce type VII collagen” includes modifying a cell to express type VII collagen from a COL7A1 gene in the genome, and modifying a cell to express type VII collagen from a COL7A1 gene in a nucleic acid construct outside the genome. Also, “a cell that is genetically modified to produce type VII collagen” includes a cell that expresses type VII collagen from a COL7A1 gene in the genome and a cell that expresses type VII collagen from a COL7A1 gene in a nucleic acid construct outside the genome.

Genetic modification of a cell can be carried out by introducing a COL7A1 gene or by correcting a mutation in the COL7A1 gene in the genome. The introduction of a COL7A1 gene can be carried out either by introducing a COL7A1 gene into the genome of the cell or by placing a nucleic acid construct comprising a COL7A1 gene in the cell so that the COL7A1 gene is expressed from the nucleic acid construct outside the genome. When a COL7A1 gene is introduced into the genome of a cell, the COL7A1 gene may be introduced at a specific site or may be introduced at random. In an embodiment, the COL7A1 gene is introduced into the COL7A1 locus of the genome, or a safe harbor such as the AAVS1 region.

The DEB patient blister-derived cell may be a cell of a patient with dystrophic epidermolysis bullosa to which the cell is to be administered (ie, an autologous cell), or a cell obtained from a subject other than the patient (ie, an allogeneic cell). The cell of a patient with dystrophic epidermolysis bullosa includes a cell that does not produce type VII collagen and a cell that produces type VII collagen with reduced function due to a mutation, and the “cell of a patient with dystrophic epidermolysis bullosa” as used herein may be any of them.

The DEB patient blister-derived cell may be any cell as long as it produces type VII collagen in the vicinity of the epidermal basement membrane when administered to a patient.

In the present disclosure, the term “cell” is used in the sense of including a cell after proliferation as needed. Proliferation of a cell can be carried out by culturing the cell. For example, “a blister-derived cell (of a patient with dystrophic epidermolysis bullosa)” includes a cell that is proliferated from a cell collected from a patient, and “a genetically modified cell” includes a cell that is proliferated from a cell obtained by genetic modification. When genetic modification is carried out, a cell may be prolifelated until the amount required for the genetic modification is obtained. Also, after genetic modification, the cell may be prolifelated until the amount required for treatment is obtained.

As used herein, the term “cell” can mean a single cell or multiple cells, depending on the context. Further, the cell may be a cell population composed of one type of cell or a cell population including a plurality of types of cells.

As used herein, the term “COL7A1 gene” means a nucleic acid sequence encoding type VII collagen, and is used to include cDNA as well as a sequence containing one or more introns (for example, a genomic sequence or a minigene). The representative nucleic acid sequence of the human COL7A1 gene (cDNA) is shown in SEQ ID NO: 1, and the representative amino acid sequence of human type VII collagen is shown in SEQ ID NO: 2. The cDNA sequence of the COL7A1 gene is disclosed in GenBank: NM_000094.3, and the genome sequence is disclosed in GenBank: AC121252.4. The sequence of a COL7A1 gene is not limited to any specific sequence as long as it encodes a functional type VII collagen (ie, a type VII collagen capable of forming anchoring fibrils).

cDNA sequence of human COL7A1 gene (8835 bp)
(SEQ ID NO: 1)
ATGACGCTGCGGCTTCTGGTGGCCGCGCTCTGCGCCGGGATCCTGGCAGA
GGCGCCCCGAGTGCGAGCCCAGCACAGGGAGAGAGTGACCTGCACGCGCC
TTTACGCCGCTGACATTGTGTTCTTACTGGATGGCTCCTCATCCATTGGC
CGCAGCAATTTCCGCGAGGTCCGCAGCTTTCTCGAAGGGCTGGTGCTGCC
TTTCTCTGGAGCAGCCAGTGCACAGGGTGTGCGCTTTGCCACAGTGCAGT
ACAGCGATGATCCACGGACAGAGTTCGGCCTGGATGCACTTGGCTCTGGG
GGTGATGTGATCCGCGCCATCCGTGAGCTTAGCTACAAGGGGGGCAACAC
TCGCACAGGGGCTGCAATTCTCCATGTGGCTGACCATGTCTTCCTGCCCC
AGCTGGCCCGACCTGGTGTCCCCAAGGTCTGCATCCTGATCACAGACGGG
AAGTCCCAGGACCTGGTGGACACAGCTGCCCAAAGGCTGAAGGGGCAGGG
GGTCAAGCTATTTGCTGTGGGGATCAAGAATGCTGACCCTGAGGAGCTGA
AGCGAGTTGCCTCACAGCCCACCAGTGACTTCTTCTTCTTCGTCAATGAC
TTCAGCATCTTGAGGACACTACTGCCCCTCGTTTCCCGGAGAGTGTGCAC
GACTGCTGGTGGCGTGCCTGTGACCCGACCTCCGGATGACTCGACCTCTG
CTCCACGAGACCTGGTGCTGTCTGAGCCAAGCAGCCAATCCTTGAGAGTA
CAGTGGACAGCGGCCAGTGGCCCTGTGACTGGCTACAAGGTCCAGTACAC
TCCTCTGACGGGGCTGGGACAGCCACTGCCGAGTGAGCGGCAGGAGGTGA
ACGTCCCAGCTGGTGAGACCAGTGTGCGGCTGCGGGGTCTCCGGCCACTG
ACCGAGTACCAAGTGACTGTGATTGCCCTCTACGCCAACAGCATCGGGGA
GGCTGTGAGCGGGACAGCTCGGACCACTGCCCTAGAAGGGCCGGAACTGA
CCATCCAGAATACCACAGCCCACAGCCTCCTGGTGGCCTGGCGGAGTGTG
CCAGGTGCCACTGGCTACCGTGTGACATGGCGGGTCCTCAGTGGTGGGCC
CACACAGCAGCAGGAGCTGGGCCCTGGGCAGGGTTCAGTGTTGCTGCGTG
ACTTGGAGCCTGGCACGGACTATGAGGTGACCGTGAGCACCCTATTTGGC
CGCAGTGTGGGGCCCGCCACTTCCCTGATGGCTCGCACTGACGCTTCTGT
TGAGCAGACCCTGCGCCCGGTCATCCTGGGCCCCACATCCATCCTCCTTT
CCTGGAACTTGGTGCCTGAGGCCCGTGGCTACCGGTTGGAATGGCGGCGT
GAGACTGGCTTGGAGCCACCGCAGAAGGTGGTACTGCCCTCTGATGTGAC
CCGCTACCAGTTGGATGGGCTGCAGCCGGGCACTGAGTACCGCCTCACAC
TCTACACTCTGCTGGAGGGCCACGAGGTGGCCACCCCTGCAACCGTGGTT
CCCACTGGACCAGAGCTGCCTGTGAGCCCTGTAACAGACCTGCAAGCCAC
CGAGCTGCCCGGGCAGCGGGTGCGAGTGTCCTGGAGCCCAGTCCCTGGTG
CCACCCAGTACCGCATCATTGTGCGCAGCACCCAGGGGGTTGAGCGGACC
CTGGTGCTTCCTGGGAGTCAGACAGCATTCGACTTGGATGACGTTCAGGC
TGGGCTTAGCTACACTGTGCGGGTGTCTGCTCGAGTGGGTCCCCGTGAGG
GCAGTGCCAGTGTCCTCACTGTCCGCCGGGAGCCGGAAACTCCACTTGCT
GTTCCAGGGCTGCGGGTTGTGGTGTCAGATGCAACGCGAGTGAGGGTGGC
CTGGGGACCCGTCCCTGGAGCCAGTGGATTTCGGATTAGCTGGAGCACAG
GCAGTGGTCCGGAGTCCAGCCAGACACTGCCCCCAGACTCTACTGCCACA
GACATCACAGGGCTGCAGCCTGGAACCACCTACCAGGTGGCTGTGTCGGT
ACTGCGAGGCAGAGAGGAGGGCCCTGCTGCAGTCATCGTGGCTCGAACGG
ACCCACTGGGCCCAGTGAGGACGGTCCATGTGACTCAGGCCAGCAGCTCA
TCTGTCACCATTACCTGGACCAGGGTTCCTGGCGCCACAGGATACAGGGT
TTCCTGGCACTCAGCCCACGGCCCAGAGAAATCCCAGTTGGTTTCTGGGG
AGGCCACGGTGGCTGAGCTGGATGGACTGGAGCCAGATACTGAGTATACG
GTGCATGTGAGGGCCCATGTGGCTGGCGTGGATGGGCCCCCTGCCTCTGT
GGTTGTGAGGACTGCCCCTGAGCCTGTGGGTCGTGTGTCGAGGCTGCAGA
TCCTCAATGCTTCCAGCGACGTTCTACGGATCACCTGGGTAGGGGTCACT
GGAGCCACAGCTTACAGACTGGCCTGGGGCCGGAGTGAAGGCGGCCCCAT
GAGGCACCAGATACTCCCAGGAAACACAGACTCTGCAGAGATCCGGGGTC
TCGAAGGTGGAGTCAGCTACTCAGTGCGAGTGACTGCACTTGTCGGGGAC
CGCGAGGGCACACCTGTCTCCATTGTTGTCACTACGCCGCCTGAGGCTCC
GCCAGCCCTGGGGACGCTTCACGTGGTGCAGCGCGGGGAGCACTCGCTGA
GGCTGCGCTGGGAGCCGGTGCCCAGAGCGCAGGGCTTCCTTCTGCACTGG
CAACCTGAGGGTGGCCAGGAACAGTCCCGGGTCCTGGGGCCCGAGCTCAG
CAGCTATCACCTGGACGGGCTGGAGCCAGCGACACAGTACCGCGTGAGGC
TGAGTGTCCTAGGGCCAGCTGGAGAAGGGCCCTCTGCAGAGGTGACTGCG
CGCACTGAGTCACCTCGTGTTCCAAGCATTGAACTACGTGTGGTGGACAC
CTCGATCGACTCGGTGACTTTGGCCTGGACTCCAGTGTCCAGGGCATCCA
GCTACATCCTATCCTGGCGGCCACTCAGAGGCCCTGGCCAGGAAGTGCCT
GGGTCCCCGCAGACACTTCCAGGGATCTCAAGCTCCCAGCGGGTGACAGG
GCTAGAGCCTGGCGTCTCTTACATCTTCTCCCTGACGCCTGTCCTGGATG
GTGTGCGGGGTCCTGAGGCATCTGTCACACAGACGCCAGTGTGCCCCCGT
GGCCTGGCGGATGTGGTGTTCCTACCACATGCCACTCAAGACAATGCTCA
CCGTGCGGAGGCTACGAGGAGGGTCCTGGAGCGTCTGGTGTTGGCACTTG
GGCCTCTTGGGCCACAGGCAGTTCAGGTTGGCCTGCTGTCTTACAGTCAT
CGGCCCTCCCCACTGTTCCCACTGAATGGCTCCCATGACCTTGGCATTAT
CTTGCAAAGGATCCGTGACATGCCCTACATGGACCCAAGTGGGAACAACC
TGGGCACAGCCGTGGTCACAGCTCACAGATACATGTTGGCACCAGATGCT
CCTGGGCGCCGCCAGCACGTACCAGGGGTGATGGTTCTGCTAGTGGATGA
ACCCTTGAGAGGTGACATATTCAGCCCCATCCGTGAGGCCCAGGCTTCTG
GGCTTAATGTGGTGATGTTGGGAATGGCTGGAGCGGACCCAGAGCAGCTG
CGTCGCTTGGCGCCGGGTATGGACTCTGTCCAGACCTTCTTCGCCGTGGA
TGATGGGCCAAGCCTGGACCAGGCAGTCAGTGGTCTGGCCACAGCCCTGT
GTCAGGCATCCTTCACTACTCAGCCCCGGCCAGAGCCCTGCCCAGTGTAT
TGTCCAAAGGGCCAGAAGGGGGAACCTGGAGAGATGGGCCTGAGAGGACA
AGTTGGGCCTCCTGGCGACCCTGGCCTCCCGGGCAGGACCGGTGCTCCCG
GCCCCCAGGGGCCCCCTGGAAGTGCCACTGCCAAGGGCGAGAGGGGCTTC
CCTGGAGCAGATGGGCGTCCAGGCAGCCCTGGCCGCGCCGGGAATCCTGG
GACCCCTGGAGCCCCTGGCCTAAAGGGCTCTCCAGGGTTGCCTGGCCCTC
GTGGGGACCCGGGAGAGCGAGGACCTCGAGGCCCAAAGGGGGAGCCGGGG
GCTCCCGGACAAGTCATCGGAGGTGAAGGACCTGGGCTTCCTGGGCGGAA
AGGGGACCCTGGACCATCGGGCCCCCCTGGACCTCGTGGACCACTGGGGG
ACCCAGGACCCCGTGGCCCCCCAGGGCTTCCTGGAACAGCCATGAAGGGT
GACAAAGGCGATCGTGGGGAGCGGGGTCCCCCTGGACCAGGTGAAGGTGG
CATTGCTCCTGGGGAGCCTGGGCTGCCGGGTCTTCCCGGAAGCCCTGGAC
CCCAAGGCCCCGTTGGCCCCCCTGGAAAGAAAGGAGAAAAAGGTGACTCT
GAGGATGGAGCTCCAGGCCTCCCAGGACAACCTGGGTCTCCGGGTGAGCA
GGGCCCACGGGGACCTCCTGGAGCTATTGGCCCCAAAGGTGACCGGGGCT
TTCCAGGGCCCCTGGGTGAGGCTGGAGAGAAGGGCGAACGTGGACCCCCA
GGCCCAGCGGGATCCCGGGGGCTGCCAGGGGTTGCTGGACGTCCTGGAGC
CAAGGGTCCTGAAGGGCCACCAGGACCCACTGGCCGCCAAGGAGAGAAGG
GGGAGCCTGGTCGCCCTGGGGACCCTGCAGTGGTGGGACCTGCTGTTGCT
GGACCCAAAGGAGAAAAGGGAGATGTGGGGCCCGCTGGGCCCAGAGGAGC
TACCGGAGTCCAAGGGGAACGGGGCCCACCCGGCTTGGTTCTTCCTGGAG
ACCCTGGCCCCAAGGGAGACCCTGGAGACCGGGGTCCCATTGGCCTTACT
GGCAGAGCAGGACCCCCAGGTGACTCAGGGCCTCCTGGAGAGAAGGGAGA
CCCTGGGCGGCCTGGCCCCCCAGGACCTGTTGGCCCCCGAGGACGAGATG
GTGAAGTTGGAGAGAAAGGTGACGAGGGTCCTCCGGGTGACCCGGGTTTG
CCTGGAAAAGCAGGCGAGCGTGGCCTTCGGGGGGCACCTGGAGTTCGGGG
GCCTGTGGGTGAAAAGGGAGACCAGGGAGATCCTGGAGAGGATGGACGAA
ATGGCAGCCCTGGATCATCTGGACCCAAGGGTGACCGTGGGGAGCCGGGT
CCCCCAGGACCCCCGGGACGGCTGGTAGACACAGGACCTGGAGCCAGAGA
GAAGGGAGAGCCTGGGGACCGCGGACAAGAGGGTCCTCGAGGGCCCAAGG
GTGATCCTGGCCTCCCTGGAGCCCCTGGGGAAAGGGGCATTGAAGGGTTT
CGGGGACCCCCAGGCCCACAGGGGGACCCAGGTGTCCGAGGCCCAGCAGG
AGAAAAGGGTGACCGGGGTCCCCCTGGGCTGGATGGCCGGAGCGGACTGG
ATGGGAAACCAGGAGCCGCTGGGCCCTCTGGGCCGAATGGTGCTGCAGGC
AAAGCTGGGGACCCAGGGAGAGACGGGCTTCCAGGCCTCCGTGGAGAACA
GGGCCTCCCTGGCCCCTCTGGTCCCCCTGGATTACCGGGAAAGCCAGGCG
AGGATGGCAAACCTGGCCTGAATGGAAAAAACGGAGAACCTGGGGACCCT
GGAGAAGACGGGAGGAAGGGAGAGAAAGGAGATTCAGGCGCCTCTGGGAG
AGAAGGTCGTGATGGCCCCAAGGGTGAGCGTGGAGCTCCTGGTATCCTTG
GACCCCAGGGGCCTCCAGGCCTCCCAGGGCCAGTGGGCCCTCCTGGCCAG
GGTTTTCCTGGTGTCCCAGGAGGCACGGGCCCCAAGGGTGACCGTGGGGA
GACTGGATCCAAAGGGGAGCAGGGCCTCCCTGGAGAGCGTGGCCTGCGAG
GAGAGCCTGGAAGTGTGCCGAATGTGGATCGGTTGCTGGAAACTGCTGGC
ATCAAGGCATCTGCCCTGCGGGAGATCGTGGAGACCTGGGATGAGAGCTC
TGGTAGCTTCCTGCCTGTGCCCGAACGGCGTCGAGGCCCCAAGGGGGACT
CAGGCGAACAGGGCCCCCCAGGCAAGGAGGGCCCCATCGGCTTTCCTGGA
GAACGCGGGCTGAAGGGCGACCGTGGAGACCCTGGCCCTCAGGGGCCACC
TGGTCTGGCCCTTGGGGAGAGGGGCCCCCCCGGGCCTTCCGGCCTTGCCG
GGGAGCCTGGAAAGCCTGGTATTCCCGGGCTCCCAGGCAGGGCTGGGGGT
GTGGGAGAGGCAGGAAGGCCAGGAGAGAGGGGAGAACGGGGAGAGAAAGG
AGAACGTGGAGAACAGGGCAGAGATGGCCCTCCTGGACTCCCTGGAACCC
CTGGGCCCCCCGGACCCCCTGGCCCCAAGGTGTCTGTGGATGAGCCAGGT
CCTGGACTCTCTGGAGAACAGGGACCCCCTGGACTCAAGGGTGCTAAGGG
GGAGCCGGGCAGCAATGGTGACCAAGGTCCCAAAGGAGACAGGGGTGTGC
CAGGCATCAAAGGAGACCGGGGAGAGCCTGGACCGAGGGGTCAGGACGGC
AACCCGGGTCTACCAGGAGAGCGTGGTATGGCTGGGCCTGAAGGGAAGCC
GGGTCTGCAGGGTCCAAGAGGCCCCCCTGGCCCAGTGGGTGGTCATGGAG
ACCCTGGACCACCTGGTGCCCCGGGTCTTGCTGGCCCTGCAGGACCCCAA
GGACCTTCTGGCCTGAAGGGGGAGCCTGGAGAGACAGGACCTCCAGGACG
GGGCCTGACTGGACCTACTGGAGCTGTGGGACTTCCTGGACCCCCCGGCC
CTTCAGGCCTTGTGGGTCCACAGGGGCTCCAGGTTTGCCTGGACAAGTGG
GGGTAGACAGGGAAGCCGGGAGCCCCAGGTCGAGATGGTGCCAGTGGAAA
AGATGGAGACAGAGGGAGCCCTGGTGTGCCAGGGTCACCAGGTCTGCCTG
GCCCTGTCGGACCTAAAGGAGAACCTGGCCCCACGGGGGCCCCTGGACAG
GCTGTGGTCGGGCTCCCTGGAGCAAAGGGAGAGAAGGGAGCCCCTGGAGG
CCTTGCTGGAGACCTGGTGGGTGAGCCGGGAGCCAAAGGTGACCGAGGAC
TGCCAGGGCCGCGAGGCGAGAAGGGTGAAGCTGGCCGTGCAGGGGAGCCC
GGAGACCCTGGGGAAGATGGTCAGAAAGGGGCTCCAGGACCCAAAGGTTT
CAAGGGTGACCCAGGAGTCGGGGTCCCGGGCTCCCCTGGGCCTCCTGGCC
CTCCAGGTGTGAAGGGAGATCTGGGCCTCCCTGGCCTGCCCGGTGCTCCT
GGTGTTGTTGGGTTCCCGGGTCAGACAGGCCCTCGAGGAGAGATGGGTCA
GCCAGGCCCTAGTGGAGAGCGGGGTCTGGCAGGCCCCCCAGGGAGAGAAG
GAATCCCAGGACCCCTGGGGCCACCTGGACCACCGGGGTCAGTGGGACCA
CCTGGGGCCTCTGGACTCAAAGGAGACAAGGGAGACCCTGGAGTAGGGCT
GCCTGGGCCCCGAGGCGAGCGTGGGGAGCCAGGCATCCGGGGTGAAGATG
GCCGCCCCGGCCAGGAGGGACCCCGAGGACTCACGGGGCCCCCTGGCAGC
AGGGGAGAGCGTGGGGAGAAGGGTGATGTTGGGAGTGCAGGACTAAAGGG
TGACAAGGGAGACTCAGCTGTGATCCTGGGGCCTCCAGGCCCACGGGGTG
CCAAGGGGGACATGGGTGAACGAGGGCCTCGGGGCTTGGATGGTGACAAA
GGACCTCGGGGAGACAATGGGGACCCTGGTGACAAGGGCAGCAAGGGAGA
GCCTGGTGACAAGGGCTCAGCCGGGTTGCCAGGACTGCGTGGACTCCTGG
GACCCCAGGGTCAACCTGGTGCAGCAGGGATCCCTGGTGACCCGGGATCC
CCAGGAAAGGATGGAGTGCCTGGTATCCGAGGAGAAAAAGGAGATGTTGG
CTTCATGGGTCCCCGGGGCCTCAAGGGTGAACGGGGAGTGAAGGGAGCCT
GTGGCCTTGATGGAGAGAAGGGAGACAAGGGAGAAGCTGGTCCCCCAGGC
CGCCCCGGGCTGGCAGGACACAAAGGAGAGATGGGGGAGCCTGGTGTGCC
GGGCCAGTCGGGGGCCCCTGGCAAGGAGGGCCTGATCGGTCCCAAGGGTG
ACCGAGGCTTTGACGGGCAGCCAGGCCCCAAGGGTGACCAGGGCGAGAAA
GGGGAGCGGGGAACCCCAGGAATTGGGGGCTTCCCAGGCCCCAGTGGAAA
TGATGGCTCTGCTGGTCCCCCAGGGCCACCTGGCAGTGTTGGTCCCAGAG
GCCCCGAAGGACTTCAGGGCCAGAAGGGTGAGCGAGGTCCCCCCGGAGAG
AGAGTGGTGGGGGCTCCTGGGGTCCCTGGAGCTCCTGGCGAGAGAGGGGA
GCAGGGGCGGCCAGGGCCTGCCGGTCCTCGAGGCGAGAAGGGAGAAGCTG
CACTGACGGAGGATGACATCCGGGGCTTTGTGCGCCAAGAGATGAGTCAG
CACTGTGCCTGCCAGGGCCAGTTCATCGCATCTGGATCACGACCCCTCCC
TAGTTATGCTGCAGACACTGCCGGCTCCCAGCTCCATGCTGTGCCTGTGC
TCCGCGTCTCTCATGCAGAGGAGGAAGAGCGGGTACCCCCTGAGGATGAT
GAGTACTCTGAATACTCCGAGTATTCTGTGGAGGAGTACCAGGACCCTGA
AGCTCCTTGGGATAGTGATGACCCCTGTTCCCTGCCACTGGATGAGGGCT
CCTGCACTGCCTACACCCTGCGCTGGTACCATCGGGCTGTGACAGGCAGC
ACAGAGGCCTGTCACCCTTTTGTCTATGGTGGCTGTGGAGGGAATGCCAA
CCGTTTTGGGACCCGTGAGGCCTGCGAGCGCCGCTGCCCACCCCGGGTGG
TCCAGAGCCAGGGGACAGGTACTGCCCAGGACTGA
Amino acid sequence of human type VII collagen
(2944 AA)
(SEQ ID NO: 2)
MTLRLLVAALCAGILAEAPRVRAQHRERVTCTRLYAADIVFLLDGSSSIG
RSNFREVRSFLEGLVLPFSGAASAQGVRFATVQYSDDPRTEFGLDALGSG
GDVIRAIRELSYKGGNTRTGAAILHVADHVFLPQLARPGVPKVCILITDG
KSQDLVDTAAQRLKGQGVKLFAVGIKNADPEELKRVASQPTSDFFFFVND
FSILRTLLPLVSRRVCTTAGGVPVTRPPDDSTSAPRDLVLSEPSSQSLRV
QWTAASGPVTGYKVQYTPLTGLGQPLPSERQEVNVPAGETSVRLRGLRPL
TEYQVTVIALYANSIGEAVSGTARTTALEGPELTIQNTTAHSLLVAWRSV
PGATGYRVTWRVLSGGPTQQQELGPGQGSVLLRDLEPGTDYEVTVSTLFG
RSVGPATSLMARTDASVEQTLRPVILGPTSILLSWNLVPEARGYRLEWRR
ETGLEPPQKVVLPSDVTRYQLDGLQPGTEYRLTLYTLLEGHEVATPATVV
PTGPELPVSPVTDLQATELPGQRVRVSWSPVPGATQYRIIVRSTQGVERT
LVLPGSQTAFDLDDVQAGLSYTVRVSARVGPREGSASVLTVRREPETPLA
VPGLRVVVSDATRVRVAWGPVPGASGFRISWSTGSGPESSQTLPPDSTAT
DITGLQPGTTYQVAVSVLRGREEGPAAVIVARTDPLGPVRTVHVTQASSS
SVTITWTRVPGATGYRVSWHSAHGPEKSQLVSGEATVAELDGLEPDTEYT
VHVRAHVAGVDGPPASVVVRTAPEPVGRVSRLQILNASSDVLRITWVGVT
GATAYRLAWGRSEGGPMRHQILPGNTDSAEIRGLEGGVSYSVRVTALVGD
REGTPVSIVVTTPPEAPPALGTLHVVQRGEHSLRLRWEPVPRAQGFLLHW
QPEGGQEQSRVLGPELSSYHLDGLEPATQYRVRLSVLGPAGEGPSAEVTA
RTESPRVPSIELRVVDTSIDSVTLAWTPVSRASSYILSWRPLRGPGQEVP
GSPQTLPGISSSQRVTGLEPGVSYIFSLTPVLDGVRGPEASVTQTPVCPR
GLADVVFLPHATQDNAHRAEATRRVLERLVLALGPLGPQAVQVGLLSYSH
RPSPLFPLNGSHDLGIILQRIRDMPYMDPSGNNLGTAVVTAHRYMLAPDA
PGRRQHVPGVMVLLVDEPLRGDIFSPIREAQASGLNVVMLGMAGADPEQL
RRLAPGMDSVQTFFAVDDGPSLDQAVSGLATALCQASFTTQPRPEPCPVY
CPKGQKGEPGEMGLRGQVGPPGDPGLPGRTGAPGPQGPPGSATAKGERGF
PGADGRPGSPGRAGNPGTPGAPGLKGSPGLPGPRGDPGERGPRGPKGEPG
APGQVIGGEGPGLPGRKGDPGPSGPPGPRGPLGDPGPRGPPGLPGTAMKG
DKGDRGERGPPGPGEGGIAPGEPGLPGLPGSPGPQGPVGPPGKKGEKGDS
EDGAPGLPGQPGSPGEQGPRGPPGAIGPKGDRGFPGPLGEAGEKGERGPP
GPAGSRGLPGVAGRPGAKGPEGPPGPTGRQGEKGEPGRPGDPAVVGPAVA
GPKGEKGDVGPAGPRGATGVQGERGPPGLVLPGDPGPKGDPGDRGPIGLT
GRAGPPGDSGPPGEKGDPGRPGPPGPVGPRGRDGEVGEKGDEGPPGDPGL
PGKAGERGLRGAPGVRGPVGEKGDQGDPGEDGRNGSPGSSGPKGDRGEPG
PPGPPGRLVDTGPGAREKGEPGDRGQEGPRGPKGDPGLPGAPGERGIEGF
RGPPGPQGDPGVRGPAGEKGDRGPPGLDGRSGLDGKPGAAGPSGPNGAAG
KAGDPGRDGLPGLRGEQGLPGPSGPPGLPGKPGEDGKPGLNGKNGEPGDP
GEDGRKGEKGDSGASGREGRDGPKGERGAPGILGPQGPPGLPGPVGPPGQ
GFPGVPGGTGPKGDRGETGSKGEQGLPGERGLRGEPGSVPNVDRLLETAG
IKASALREIVETWDESSGSFLPVPERRRGPKGDSGEQGPPGKEGPIGFPG
ERGLKGDRGDPGPQGPPGLALGERGPPGPSGLAGEPGKPGIPGLPGRAGG
VGEAGRPGERGERGEKGERGEQGRDGPPGLPGTPGPPGPPGPKVSVDEPG
PGLSGEQGPPGLKGAKGEPGSNGDQGPKGDRGVPGIKGDRGEPGPRGQDG
NPGLPGERGMAGPEGKPGLQGPRGPPGPVGGHGDPGPPGAPGLAGPAGPQ
GPSGLKGEPGETGPPGRGLTGPTGAVGLPGPPGPSGLVGPQGSPGLPGQV
GETGKPGAPGRDGASGKDGDRGSPGVPGSPGLPGPVGPKGEPGPTGAPGQ
AVVGLPGAKGEKGAPGGLAGDLVGEPGAKGDRGLPGPRGEKGEAGRAGEP
GDPGEDGQKGAPGPKGFKGDPGVGVPGSPGPPGPPGVKGDLGLPGLPGAP
GVVGFPGQTGPRGEMGQPGPSGERGLAGPPGREGIPGPLGPPGPPGSVGP
PGASGLKGDKGDPGVGLPGPRGERGEPGIRGEDGRPGQEGPRGLTGPPGS
RGERGEKGDVGSAGLKGDKGDSAVILGPPGPRGAKGDMGERGPRGLDGDK
GPRGDNGDPGDKGSKGEPGDKGSAGLPGLRGLLGPQGQPGAAGIPGDPGS
PGKDGVPGIRGEKGDVGFMGPRGLKGERGVKGACGLDGEKGDKGEAGPPG
RPGLAGHKGEMGEPGVPGQSGAPGKEGLIGPKGDRGFDGQPGPKGDQGEK
GERGTPGIGGFPGPSGNDGSAGPPGPPGSVGPRGPEGLQGQKGERGPPGE
RVVGAPGVPGAPGERGEQGRPGPAGPRGEKGEAALTEDDIRGFVRQEMSQ
HCACQGQFIASGSRPLPSYAADTAGSQLHAVPVLRVSHAEEEERVPPEDD
EYSEYSEYSVEEYQDPEAPWDSDDPCSLPLDEGSCTAYTLRWYHRAVTGS
TEACHPFVYGGCGGNANRFGTREACERRCPPRVVQSQGTGTAQD*

In an embodiment, the COL7A1 gene comprises or consists of a nucleic acid sequence having 70%, 80%, 85%, 90%, 95%, 96%, 97%, 98% or 99% or more sequence identity with the nucleic acid sequence of SEQ ID NO: 1. In a different embodiment, the COL7A1 gene comprises or consists of a nucleic acid sequence wherein 1 to 30, 1 to 20, 1 to 10, 1 to 5, 1 to 3, 1 to 2 or 1 base(s) is inserted, deleted, substituted, or added with respect to the nucleic acid sequence of SEQ ID NO: 1. In a further embodiment, the COL7A1 gene comprises or consists of the nucleic acid sequence of SEQ ID NO: 1.

In an embodiment, the type VII collagen comprises or consists of an amino acid sequence having 70%, 80%, 85%, 90%, 95%, 96%, 97%, 98% or 99% or more sequence identity with the amino acid sequence of SEQ ID NO: 2. In a different embodiment, the type VII collagen comprises or consists of an amino acid sequence wherein 1 to 30, 1 to 20, 1 to 10, 1 to 5, 1 to 3, 1 to 2 or 1 amino acid residue(s) is inserted, deleted, substituted, or added with respect to the amino acid sequence of SEQ ID NO: 2. In a further embodiment, the type VII collagen comprises or consists of the amino acid sequence of SEQ ID NO: 2.

In an embodiment, the COL7A1 gene comprises or consists of a nucleic acid sequence that encodes an amino acid sequence having 70%, 80%, 85%, 90%, 95%, 96%, 97%, 98% or 99% or more sequence identity with the amino acid sequence of SEQ ID NO: 2. In a different embodiment, the COL7A1 gene comprises or consists of a nucleic acid sequence that encodes an amino acid sequence wherein 1 to 30, 1 to 20, 1 to 10, 1 to 5, 1 to 3, 1 to 2 or 1 amino acid residue(s) is inserted, deleted, substituted, or added with respect to the amino acid sequence of SEQ ID NO: 2.

As used herein, the term “sequence identity” with respect to a nucleic acid sequence or an amino acid sequence means the proportion of bases or amino acid residues that match between two sequences that are optimally aligned (aligned to be maximally matched) over the entire region of the sequence to be compared. The sequence to be compared may have an insertion, an addition or a deletion (eg, a gap) in the optimal alignment of the two sequences. The sequence identity can be calculated using a program such as FASTA, BLAST, or CLUSTAL W provided in a public database (for example, DDBJ (http://www.ddbj.nig.ac.jp)). Alternatively, the sequence identity can be obtained using a commercially available sequence analysis software (for example, Vector NTI® software, GENETYX® ver. 12).

The cell may be genetically modified by any method. In an embodiment, the cell is genetically modified by genome editing such as the CRISPR system (eg, CRISPR/Cas9, CRISPR/Cpf1), TALEN, or ZFN. In a different embodiment, the cell is genetically modified with a viral vector such as a retroviral vector, lentiviral vector, adenoviral vector, or adeno-associated viral vector. In a further embodiment, the cell is genetically modified with CRISPR/Cas9. In a further embodiment, the cell is genetically modified with a retroviral vector or a lentiviral vector.

In genome editing, causing cleavage in the genome and introducing a donor vector comprising a sequence of interest into the cell can insert the sequence of interest into the cleavage site of the genome. The sequence to be inserted into the genome can be a COL7A1 gene or a sequence to be replaced with a portion containing a mutation in the COL7A1 gene (for example, a partial sequence of a COL7A1 gene). In addition to the sequence of interest, the donor vector may comprise a regulatory sequence such as a promoter or enhancer that controls the expression of the sequence of interest, or other elements such as a drug resistance gene for cell selection, and also may comprise, at both ends, sequences homogeneous to both ends of the insertion site of the genome. The donor vector can be introduced into a desired site as a result of non-homologous end binding or homologous recombination. As the donor vector, a plasmid, an adeno-associated viral vector, an integrase-deficient lentiviral vector, or any of other viral vectors can be used.

In the CRISPR system, an endonuclease such as Cas9 or Cas12 (eg, Cas12a (also called Cpf1), Cas12b, Cas12e) recognizes a specific base sequence, called PAM sequence, and the double strand of the target DNA is cleaved by the action of the endonuclease. When the endonuclease is Cas9, it cleaves about 3-4 bases upstream of the PAM sequence. Examples of endonucleases include Cas9 of S. pyogenes, S. aureus, N. meningitidis, S. thermophilus, or T. denticola, and Cpfl of L. bacterium ND2006 or Acidaminococcus sp. BV3L6. The PAM sequence varies depending on the endonuclease, and the PAM sequence of Cas9 in S. pyogenes is NGG, for example. A gRNA comprises a sequence of about 20 bases upstream of the PAM sequence (target sequence) or a sequence complementary thereto on the 5′ end side, and plays a role of recruiting an endonuclease to the target sequence. The sequences other than the target sequence (or a sequence complementary thereto) of a gRNA can be appropriately determined by those skilled in the art depending on the endonuclease to be used. A gRNA may comprises a crRNA (CRISPR RNA), which comprises the target sequence or a sequence complementary thereto and is responsible for the sequence specificity of the gRNA, and a tracrRNA (Trans-activating crRNA), which contributes to the formation of a complex with Cas9 by forming a double strand. The crRNA and tracrRNA may exist as separate molecules. When the endonuclease is Cpf1, the crRNA alone functions as a gRNA. In the present specification, a gRNA comprising elements necessary for the function as a gRNA on a single strand may be particularly referred to as a sgRNA. The gRNA sequence can be determined by a tool available for target sequence selection and gRNA design, such as CRISPRdirect (https://crispr.dbcls.jp/).

A vector comprising a nucleic acid sequence encoding a gRNA and a nucleic acid sequence encoding an endonuclease may be introduced into and expressed in a cell, or a gRNA and an endonuclease protein that have been prepared extracellularly may be introduced into a cell. The endonuclease may include a nuclear localization signal. The nucleic acid sequence encoding a gRNA and the nucleic acid sequence encoding an endonuclease may be present on different vectors. Methods for introducing the vector, gRNA, and endonuclease into a cell include, but are not limited to, lipofection, electroporation, microinjection, calcium phosphate method, and DEAE-dextran method.

In an embodiment, a gRNA that can be used for the introduction of a COL7A1 gene into the genome comprises any of the sequences of SEQ ID NOs: 3 to 5 or a sequence complementary thereto.

In the case of viral vectors, a COL7A1 gene can be introduced into the genome of a cell when a retroviral vector or a lentiviral vector having integrase activity is used. Alternatively, an integrase-deficient retroviral or lentiviral vector may be used. Integrase-deficient vectors lack integrase activity, for example, due to a mutation in the integrase gene. When an integrase-deficient vector, or an adenoviral vector or an adeno-associated viral vector is used, the sequence incorporated into the vector is not usually introduced into the genome of a cell. For example, when a COL7A1 gene is incorporated into an integrase-deficient lentiviral vector or an adenoviral vector, type VII collagen is expressed from the COL7A1 gene of the vector existing in the cell (in the nucleus).

A viral vector comprises a sequence encoding a COL7A1 gene and may contain a regulatory sequence such as a promoter or enhancer that controls the expression of the COL7A1 gene and other elements such as a drug resistance gene for cell selection. A viral vector may be prepared by any method known in the art. For example, a retroviral or lentiviral vector can be prepared by introducing a viral vector plasmid comprising LTR sequences at both ends (5′ LTR and 3′ LTR), a packaging signal, and a sequence of interest into a packaging cell with one or more plasmid vectors expressing structural proteins of the virus, such as Gag, Pol, and Env, or into a packaging cell that expresses such structural proteins. Examples of packaging cells include, but are not limited to, 293T cells, 293 cells, HeLa cells, COS1 cells, and COS7 cells. The viral vector may be pseudotyped and may express an envelope protein such as the vesicular stomatitis virus G protein (VSV-G). The sequence of interest can be introduced into a target cell by infecting the target cell with a viral vector thus prepared.

In an embodiment, the viral vector is a lentiviral vector. Examples of lentiviral vectors include, but are not limited to, HIV (human immunodeficiency virus) (for example, HIV-1 and HIV-2), SIV (simian immunodeficiency virus), FIV (feline immunodeficiency virus), MVV (Maedi-Visna virus), EV1 (Maedi-Visna-like virus), EIAV (equine infectious anemia virus), and CAEV (caprine arthritis encephalitis virus). In an embodiment, the lentiviral vector is HIV.

As an example, a lentiviral vector can be prepared as follows. First, a viral vector plasmid encoding the viral genome, one or more plasmid vectors expressing Gag, Pol, and Rev (and optionally Tat), and one or more plasmid vectors expressing envelope proteins such as VSV-G are introduced into a packaging cell. The viral vector plasmid comprises LTR sequences at both ends (5′ LTR and 3′ LTR), a packaging signal, and a COL7A1 gene and a promoter that controls its expression (eg, CMV promoter, CAG promoter, EF1α promoter, PGK promoter, or hCEF promoter). The 5′ LTR functions as a promoter that induces transcription of the viral RNA genome, but may be replaced with a different promoter, such as CMV promoter, to enhance the expression of the RNA genome. Within the cell, the viral RNA genome is transcribed from the vector plasmid and packaged to form a viral core. The viral core is transported to the cell membrane of the packaging cell, encapsulated in the cell membrane, and released as a viral particle from the packaging cell. The released virus particle can be recovered from the culture supernatant of the packaging cell. For example, the virus particle can be recovered by any of conventional purification methods such as centrifugation, filter filtration, and column purification. A lentiviral vector can also be prepared by using a kit such as Lentiviral High Titer Packaging Mix, Lenti-X™ Packaging Single Shots (Takara Bio Inc.), and ViraSafe™ Lentivirus Complete Expression System (Cell Biolabs Inc.). An adeno-associated viral vector can be prepared by using a kit such as AAVpro® Helper Free System (Takara Bio Inc.).

In one aspect, the present disclosure provides a plasmid comprising an EF1α promoter and a COL7A1 gene located downstream of the EF1α promoter that is used for producing a lentiviral vector. In a further aspect, the disclosure provides a lentiviral vector comprising an EF1α promoter and a COL7A1 gene located downstream of the EF1α promoter.

A representative sequence of the EF1α promoter is shown in SEQ ID NO:6.

EF1a promoter
(SEQ ID NO: 6)
GTGAGGCTCCGGTGCCCGTCAGTGGGCAGAGCGCACATCGCCCACAGTCC
CCGAGAAGTTGGGGGGAGGGGTCGGCAATTGAACCGGTGCCTAGAGAAGG
TGGCGCGGGGTAAACTGGGAAAGTGATGTCGTGTACTGGCTCCGCCTTTT
TCCCGAGGGTGGGGGAGAACCGTATATAAGTGCAGTAGTCGCCGTGAACG
TTCTTTTTCGCAACGGGTTTGCCGCCAGAACACAGGTAAGTGCCGTGTGT
GGTTCCCGCGGGCCTGGCCTCTTTACGGGTTATGGCCCTTGCGTGCCTTG
AATTACTTCCACGCCCCTGGCTGCAGTACGTGATTCTTGATCCCGAGCTT
CGGGTTGGAAGTGGGTGGGAGAGTTCGAGGCCTTGCGCTTAAGGAGCCCC
TTCGCCTCGTGCTTGAGTTGAGGCCTGGCTTGGGCGCTGGGGCCGCCGCG
TGCGAATCTGGTGGCACCTTCGCGCCTGTCTCGCTGCTTTCGATAAGTCT
CTAGCCATTTAAAATTTTTGATGACCTGCTGCGACGCTTTTTTTCTGGCA
AGATAGTCTTGTAAATGCGGGCCAAGATCTGCACACTGGTATTTCGGTTT
TTGGGGCCGCGGGCGGCGACGGGGCCCGTGCGTCCCAGCGCACATGTTCG
GCGAGGCGGGGCCTGCGAGCGCGGCCACCGAGAATCGGACGGGGGTAGTC
TCAAGCTGGCCGGCCTGCTCTGGTGCCTGGCCTCGCGCCGCCGTGTATCG
CCCCGCCCTGGGCGGCAAGGCTGGCCCGGTCGGCACCAGTTGCGTGAGCG
GAAAGATGGCCGCTTCCCGGCCCTGCTGCAGGGAGCTCAAAATGGAGGAC
GCGGCGCTCGGGAGAGCGGGCGGGTGAGTCACCCACACAAAGGAAAAGGG
CCTTTCCGTCCTCAGCCGTCGCTTCATGTGACTCCACGGAGTACCGGGCG
CCGTCCAGGCACCTCGATTAGTTCTCGAGCTTTTGGAGTACGTCGTCTTT
AGGTTGGGGGGAGGGGTTTTATGCGATGGAGTTTCCCCACACTGAGTGGG
TGGAGACTGAAGTTAGGCCAGCTTGGCACTTGATGTAATTCTCCTTGGAA
TTTGCCCTTTTTGAGTTTGGATCTTGGTTCATTCTCAAGCCTCAGACAGT
GGTTCAAAGTTTTTTTCTTCCATTTCAGGTGTCGTGAA

In an embodiment, the EF1α promoter comprises or consists of a nucleic acid sequence having 70%, 80%, 85%, 90%, 95%, 96%, 97%, 98% or 99% or more sequence identity with the nucleic acid sequence of SEQ ID NO: 6. In a different embodiment, the EF1α promoter comprises or consists of a nucleic acid sequence wherein 1 to 30, 1 to 20, 1 to 10, 1 to 5, 1 to 3, 1 to 2 or 1 base(s) is inserted, deleted, substituted, or added with respect to the nucleic acid sequence of SEQ ID NO: 6. In a further embodiment, the EF1α promoter comprises or consists of the nucleic acid sequence of SEQ ID NO: 6.

A cell into which a sequence of interest has been introduced can be detected by Southern blotting or PCR. The sequence of interest need only be introduced into at least one of the alleles.

In an embodiment of the composition of the present disclosure, the DEB patient blister-derived cell is the most abundant cell in the composition. In a further embodiment, the DEB patient blister-derived cell accounts for 70%, 80%, 85%, 90%, 95%, 96%, 97%, 98% or 99% or more of cells comprised in the composition. In a further embodiment, the composition of the present disclosure is substantially free of cells other than the DEB patient blister-derived cell. The expression “substantially free of cells other than the DEB patient blister-derived cell” means that the composition only comprises a cell obtained by a method that is substantially identical to the method for obtaining the DEB patient blister-derived cell as described herein.

The number of cells comprised in a composition is an amount required to exert a desired effect (also referred to herein as an effective amount), and it is appropriately determined by those skilled in the art in consideration of factors such as the age, body weight, and medical condition of the patient, the type of cells and method for genetic modification. The number of cells is not limited to, but can be, for example, 1 cell to 1×10⁷ cells, 1×10 cells to 1×10⁷ cells, 1×10² cells to 1×10⁷ cells, 1×10³ cells to 1×10⁷ cells, 1×10⁴ cells to 1×10⁷ cells, 1×10⁵ cells to 1×10⁷ cells, 1×10⁵ cells to 5×10⁶ cells, 5×10⁵ cells to 1×10⁶ cells, or 1×10⁵ cells to 1×10⁶ cells. The composition may comprise a pharmaceutically acceptable carrier and/or an additive in addition to the cell. Examples of pharmaceutically acceptable carriers include water, medium, saline, infusion containing glucose, D-sorbitol, D-mannitol or others, and phosphate buffered saline (PBS). Examples of additives include solubilizers, stabilizers, and preservatives. The dosage form of the composition is not particularly limited to, but can be a parenteral preparation such as an injection. Examples of injections include solution injections, suspension injections, emulsion injections, and injections to be prepared before use. The composition may be frozen and may contain a cryoprotectant such as DMSO, glycerol, polyvinylpyrrolidone, polyethylene glycol, dextran, or sucrose.

The composition of the present disclosure can be administered systemically or topically. In an embodiment, the composition is administered to an affected area of a patient with dystrophic epidermolysis bullosa. As used herein, the affected area means a blister or an area in the vicinity of a blister. In a further embodiment, the composition is administered intradermally at the site of a blister or administered into a blister. In a further embodiment, the composition is administered into a blister. In the present specification, administration into a blister means administration to the space under the epidermis of a blister. The composition is preferably administered into the space formed between the epidermis and the dermis due to detachment of the epidermis from the dermis. The composition is more preferably administered into the space formed between the basement membrane of the epidermis and the dermis due to detachment of the basement membrane from the dermis. For example, the composition can be administered into a blister by piercing the blister with an injection needle of a syringe containing the composition in the syringe and ejecting the composition from the tip of the injection needle positioned in the space formed between the epidermis and the dermis. The blister may be a blister naturally formed as a pathological condition of epidermolysis bullosa, or may be a blister artificially formed. In patients with epidermolysis bullosa, a blister can be artificially formed, for example, by pinching or rubbing the patient's skin. The intrablister administration can reduce the patient's pain compared to intradermal or subcutaneous administration, and type VII collagen can be well expressed around the basement membrane. The number of cells administered per site is an amount required to exert a desired effect (effective amount), and it is appropriately determined by those skilled in the art in consideration of factors such as the age, body weight, and medical condition of the patient, the type of cells, and method for genetic modification. The number of cells is not limited to, but can be for example, 1 cell to 1×10⁷ cells, 1×10 cells to 1×10⁷ cells, 1×10² cells to 1×10⁷ cells, 1×10³ cells to 1×10⁷ cells, 1×10⁴ cells to 1×10⁷ cells, 1×10⁵ cells to 1×10⁷ cells, 1×10⁵ cells to 5×10⁶ cells, 5×10⁵ cells to 1×10⁶ cells, or 1×10⁵ cells to 1×10⁶ cells. In an embodiment, the number of cells to be administered per blister is 1 cell to 1×10⁷ cells, 1×10 cells to 1×10⁷ cells, 1×10² cells to 1×10⁷ cells, 1×10³ cells to 1×10⁷ cells, 1×10⁴ cells to 1×10⁷ cells, 1×10⁵ cells to 1×10⁷ cells, 1×10⁵ cells to 5×10⁶ cells, 5×10⁵ cells to 1×10⁶ cells, or 1×10⁵ cells to 1×10⁶ cells. The amount to be administered per blister may be adjusted according to the size of the blister, and the above amount may be considered to be an amount for a standard blister having a diameter of 7 to 8 mm when circularly approximated. When the cells are administered into a blister, a preferred dosage is 1×10⁵ to 1×10⁷ cells per cm² of blister area, and a more preferred dosage is 5×10⁵ to 5×10⁶ cells per cm² of blister area.

Exemplary embodiments of the present invention are described below.
[1] A composition for use in the treatment of dystrophic epidermolysis bullosa, comprising a blister-derived cell of a patient with dystrophic epidermolysis bullosa that is genetically modified to produce type VII collagen.
[2] The composition according to item 1, wherein the blister-derived cell is genetically modified by introducing a COL7A1 gene.
[3] The composition according to item 2, wherein the COL7A1 gene is introduced into the genome of the blister-derived cell.
[4] The composition according to item 2 or 3, wherein the COL7A1 gene comprises a nucleic acid sequence having 70%, 80%, 85%, 90%, 95%, 96%, 97%, 98% or 99% or more sequence identity with the nucleic acid sequence of SEQ ID NO: 1, or a nucleic acid sequence that encodes an amino acid sequence having 70%, 80%, 85%, 90%, 95%, 96%, 97%, 98% or 99% or more sequence identity with the amino acid sequence of SEQ ID NO: 2.
[5] The composition according to any one of items 1 to 4, wherein the blister-derived cell has one or more characteristics selected from:
1) adherent to a solid phase;
2) positive for one or more surface markers selected from the group consisting of CD73, CD105 and CD90;
3) negative for one or more surface markers selected from the group consisting of CD45, CD34, CD11b, CD79A, HLA-DR and CD31;
4) incapable of differentiating into an osteoblast, or less capable of differentiating into an osteoblast than a bone marrow-derived mesenchymal stem cell;
5) incapable of differentiating into an adipocyte, or less capable of differentiating into an adipocyte than a bone marrow-derived mesenchymal stem cell; and
6) incapable of differentiating into a chondrocyte, or less capable of differentiating into a chondrocyte than a bone marrow-derived mesenchymal stem cell.
[6] The composition according to any one of items 1 to 5, wherein the blister-derived cell is derived from a blister fluid of the patient with dystrophic epidermolysis bullosa.
[7] The composition according to any one of items 1 to 6, wherein the blister-derived cell is the most abundant cell in the composition.
[8] The composition according to any one of items 1 to 7, wherein the blister-derived cell accounts for 70%, 80%, 85%, 90%, 95%, 96%, 97%, 98% or 99% or more of cells comprised in the composition.
[9] The composition according to any one of items 1 to 8, wherein the composition does not substantially comprise cells other than the blister-derived cell.
[10] The composition according to any one of items 1 to 9, which is to be administered to an affected area.
[11] The composition according to any one of items 1 to 10, which is to be administered into a blister.
[12] The composition according to any one of items 1 to 11, wherein the blister-derived cell is genetically modified by genome editing.
[13] The composition according to item 12, wherein the genome editing is carried out by CRISPR/Cas9.
[14] The composition according to any one of items 1 to 11, wherein the blister-derived cell is genetically modified with a viral vector.
[15] The composition according to item 14, wherein the viral vector is a retroviral vector or a lentiviral vector.
[16] The composition according to item 14 or 15, wherein the viral vector is a lentiviral vector.
[17] A method of producing a composition for use in the treatment of dystrophic epidermolysis bullosa, comprising genetically modifying a blister-derived cell of a patient with dystrophic epidermolysis bullosa to produce type VII collagen, and
preparing a composition comprising the genetically modified blister-derived cell.
[18] A method of treating dystrophic epidermolysis bullosa, comprising administering a composition comprising a blister-derived cell of a patient with dystrophic epidermolysis bullosa that is genetically modified to produce type VII collagen to the patient.
[19] The method according to item 18, wherein the blister-derived cell is genetically modified by introducing a COL7A1 gene.
[20] The method according to item 19, wherein the COL7A1 gene is introduced into the genome of the blister-derived cell.
[21] The method according to item 19 or 20, wherein the COL7A1 gene comprises a nucleic acid sequence having 70%, 80%, 85%, 90%, 95%, 96%, 97%, 98% or 99% or more sequence identity with the nucleic acid sequence of SEQ ID NO: 1, or a nucleic acid sequence that encodes an amino acid sequence having 70%, 80%, 85%, 90%, 95%, 96%, 97%, 98% or 99% or more sequence identity with the amino acid sequence of SEQ ID NO: 2.
[22] The method according to any one of items of 18 to 21, wherein the blister-derived cell is genetically modified by genome editing.
[23] The method according to item 22, wherein the genome editing is carried out by CRISPR/Cas9.
[24] The method according to any one of items 18 to 21, wherein the blister-derived cell is genetically modified with a viral vector.
[25] The method according to item 24, wherein the viral vector is a retroviral vector or a lentiviral vector.
[26] The method according to item 24 or 25, wherein the viral vector is a lentiviral vector.
[27] The method according to any one of items 18-26, further comprising, prior to the adminstering to the patient, genetically modifying a blister-derived cell to produce type VII collagen.
[28] The method according to item 17 or 27, wherein the genetically modifying comprises introducting a COL7A1 gene into the blister-derived cell.
[29] The method according to item 28, wherein the COL7A1 gene is introduced into the genome of the blister-derived cell.
[30] The method according to item 28 or 29, wherein the COL7A1 gene comprises a nucleic acid sequence having 70%, 80%, 85%, 90%, 95%, 96%, 97%, 98% or 99% or more sequence identity with the nucleic acid sequence of SEQ ID NO: 1, or a nucleic acid sequence that encodes an amino acid sequence having 70%, 80%, 85%, 90%, 95%, 96%, 97%, 98% or 99% or more sequence identity with the amino acid sequence of SEQ ID NO: 2.
[31] The method according to any one of items of 17 and 27 to 30, wherein the genetically modifying comprises genetically modifying the blister-derived cell by genome editing.
[32] The method according to item 31, wherein the genome editing is carried out by CRISPR/Cas9.
[33] The method according to any one of items 17 and 27-30, wherein the genetically modifying comprises genetically modifying the blister-derived cell with a viral vector.
[34] The method according to item 33, wherein the viral vector is a retroviral vector or a lentiviral vector.
[35] The method according to item 33 or 34, wherein the viral vector is a lentiviral vector.
[36] The method according to any one of items 17 and 27-35, further comprising, prior to the genetically modifying, obtaining the blister-derived cell from a blister of the patient with dystrophic epidermolysis bullosa.
[37] The method according to any one of items 17 to 36, wherein the blister-derived cell is derived from a blister fluid of the patient with dystrophic epidermolysis bullosa.
[38] The method according to any one of items 17 to 37, wherein the blister-derived cell is the most abundant cell in the composition.
[39] The method according to any one of items 17 to 38, wherein the blister-derived cell accounts for 70%, 80%, 85%, 90%, 95%, 96%, 97%, 98% or 99% or more of cells comprised in the composition.
[40] The method according to any one of items 17 to 39, wherein the composition does not substantially comprise cells other than the blister-derived cell.
[41] The method according to any one of items 18 to 40, wherein the composition is administered to an affected area.
[42] The method according to any one of items 18-41, wherein the composition is administered into a blister.
[43] Use of a composition comprising a blister-derived cell of a patient with dystrophic epidermolysis bullosa that is genetically modified to produce type VII collagen for the manufacture of a medicament for treating dystrophic epidermolysis bullosa.
[44] The use according to item 43, wherein the composition is to be administered into a blister.
[45] The use according to item 43 or 44, wherein the blister-

derived cell is derived from a blister fluid of the patient with dystrophic epidermolysis bullosa.
[46] Use of a composition comprising a blister-derived cell of a patient with dystrophic epidermolysis bullosa that is genetically modified to produce type VII collagen for treating dystrophic epidermolysis bullosa.
[47] The use according to item 46, wherein the composition is administered into a blister.
[48] The use according to item 46 or 47, wherein the blister-derived cell is derived from a blister fluid of the patient with dystrophic epidermolysis bullosa.
[49] A blister-derived cell of a patient with dystrophic epidermolysis bullosa that is genetically modified to produce type VII collagen for use in the treatment of dystrophic epidermolysis bullosa.
[50] The cell according to item 49, wherein the cell is to be administered into a blister.
[51] The cell according to item 49 or 50, wherein the cell is derived from a blister fluid of the patient with dystrophic epidermolysis bullosa.
[52] A gRNA comprising a sequence of any one of SEQ ID NOs: 3 to 5 or a sequence complementary thereto.
[53] A vector comprising a nucleic acid sequence encoding the gRNA of item 52.
[54] A method for producing a cell, comprising culturing a blister content of a patient with dystrophic epidermolysis bullosa on a solid phase.
[55] The method according to item 54, wherein the blister content is a blister fluid.
[56] The method according to item 54 or 55, wherein the blister content is collected from a blister of the patient and seeded in a medium without any enzymatic treatment before the culturing on the solid phase.
[57]

A cell produced by the method according to any one of items 54 to 56.
[58] The cell according to item 57, wherein the cell has one or more characteristics selected from:
1) adherent to a solid phase;
2) positive for one or more surface markers selected from the group consisting of CD73, CD105 and CD90;
3) negative for one or more surface markers selected from the group consisting of CD45, CD34, CD11b, CD79A, HLA-DR and CD31;
4) incapable of differentiating into an osteoblast, or less capable of differentiating into an osteoblast than a bone marrow-derived mesenchymal stem cell;
5) incapable of differentiating into an adipocyte, or less capable of differentiating into an adipocyte than a bone marrow-derived mesenchymal stem cell; and
6) incapable of differentiating into a chondrocyte, or less capable of differentiating into a chondrocyte than a bone marrow-derived mesenchymal stem cell.
[59] A method of producing a cell, comprising
1) culturing a blister content of a patient with dystrophic epidermolysis bullosa on a solid phase; and
2) genetically modifying the cell obtained from the culturing of 1) to produce type VII collagen.
[60] The method according to item 59, wherein the blister content is a blister fluid.
[61] The method according to item 59 or 60, wherein the blister content is collected from a blister of the patient and seeded in a medium without any enzymatic treatment before the culturing on the solid phase.
[62] A cell produced by the method according to any one of items 59 to 61.
[63] A blister-derived cell of a patient with dystrophic epidermolysis bullosa that is genetically modified to produce type VII collagen.
[64] The cell according to item 63, wherein the cell is derived from a blister fluid of the patient with dystrophic epidermolysis bullosa.
[65] The cell according to any one of items 62 to 64, wherein the cell is genetically modified by introducing a COL7A1 gene.
[66] The cell according to item 65, wherein the COL7A1 gene is introduced into the genome of the blister-derived cell.
[67] The cell according to item 66, wherein the COL7A1 gene comprises a nucleic acid sequence having 70%, 80%, 85%, 90%, 95%, 96%, 97%, 98% or 99% or more sequence identity with the nucleic acid sequence of SEQ ID NO: 1, or a nucleic acid sequence that encodes an amino acid sequence having 70%, 80%, 85%, 90%, 95%, 96%, 97%, 98% or 99% or more sequence identity with the amino acid sequence of SEQ ID NO: 2.
[68] The cell according to any one of items 62 to 67, wherein the cell is genetically modified by genome editing.
[69] The cell according to item 68, wherein the genome editing is carried out by CRISPR/Cas9.
[70] The cell according to any one of items 62-67, wherein the cell is genetically modified with a viral vector.
[71] The cell according to item 70, wherein the viral vector is a retroviral vector or a lentiviral vector.
[72] The cell according to item 70 or 71, wherein the viral vector is a lentiviral vector.
[73] The cell according to any one of items 62 to 72, wherein the cell has one or more characteristics selected from:
1) adherent to a solid phase;
2) positive for one or more surface markers selected from the group consisting of CD73, CD105 and CD90;
3) negative for one or more surface markers selected from the group consisting of CD45, CD34, CD11b, CD79A, HLA-DR and CD31;
4) incapable of differentiating into an osteoblast, or less capable of differentiating into an osteoblast than a bone marrow-derived mesenchymal stem cell;
5) incapable of differentiating into an adipocyte, or less capable of differentiating into an adipocyte than a bone marrow-derived mesenchymal stem cell; and
6) incapable of differentiating into a chondrocyte, or less capable of differentiating into a chondrocyte than a bone marrow-derived mesenchymal stem cell.
[74] A plasmid, comprising
an EF1α promoter, and
a COL7A1 gene located downstream of the EF1α promoter,
wherein the plasmid is used for producing a lentiviral vector.
[75] A lentiviral vector, comprising
an EF1α promoter, and
a COL7A1 gene located downstream of the EF1α promoter.

The present invention is described in more detail with reference to the examples hereinafter, but not limited to the embodiments described below.

### EXAMPLES

### 1. Collection of Blister-Derived Cells

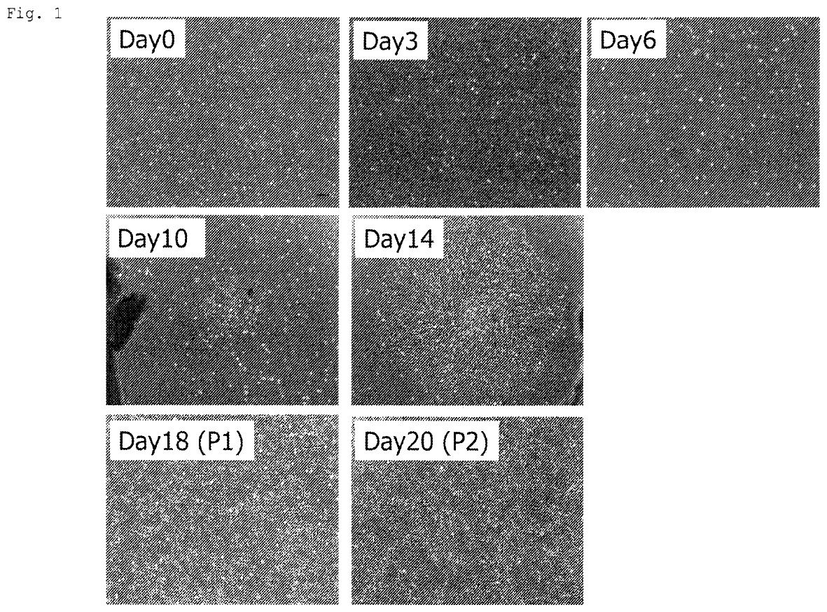

Blister fluid from a patient with dystrophic epidermolysis bullosa was collected and centrifuged at 300 g for 5 minutes. The resulting precipitate was suspended in a medium (Mesenchymal Stem Cell Growth Medium 2 (PromoCell, C-28009) supplemented with penicillin and streptomycin to a final concentration of 100 unit/mL and 100 μg/mL, respectively), and seeded in a 6-well plate coated with collagen I and cultured at 37° C. and 5% CO₂ to obtain adherent cells. The duration time from collection of the blister fluid to seeding in the medium varied from patient to patient, but was within the range of 18 minutes to 1 hour. Thereafter, medium exchange and passaging were carried out appropriately until the cells proliferated to the desired cell number (culture progress up to 20 days after initiation of culture is shown in FIG. 1). Cells obtained by such a method are hereinafter also referred to as “blister-derived cells”. Cells at the 3rd passage were used for the following surface marker analysis and differentiation induction experiment, and cells at the 3rd to 4th passage were used for gene transfer. It was confirmed that blister-derived cells obtained with a mixed medium of equal volume of Mesenchymal Stem Cell Growth Medium 2 (PromoCell, C-28009) and MSCGM Mesenchymal Stem Cell Growth Medium (Lonza, PT-3001) or Cellartis MSC Xeno-Free Culture Medium (Takara Bio, Y50200) were equivalent to the blister-derived cells obtained with the above medium. As a preliminary study, we investigated the correlation between the duration time from collecting the blister fluid from the patient's blister to inoculating it in the medium and the number of colonies formed on the plate after culturing, and found that a large number of colonies were obtained when the blister fluid was seeded within 1 hour after the collection of the blister fluid, and very few or almost no colony was obtained when the blister fluid was seeded more than 3 hours after the collection.

### 2. Characterization of Blister-Derived Cells

### a) Surface Marker Analysis (FACS)

The blister-derived cells obtained in the section 1 above and human bone marrow-derived mesenchymal stem cells (hereinafter also referred to as BM-MSCs) [purchased from PromoCell (Heidelberg, Germany) or Lonza (Basel, Switzerland)] were subjected to surface marker analysis according to the following procedure. The cells were removed from the plate with Accutase-Solution (PromoCell, C-41310), and after washed with medium, 100,000 cells each were put into two tubes based on cell count results. The cells were washed once with Flow Cytometry Staining Buffer (1×) (R&D Systems, FC001), and resuspended with 100 μl of Flow Cytometry Staining Buffer (1×). To block Fc receptors, 5 μl of Human TruStain FcX™ (BioLegend, 422301) was added and allowed to react on ice for 10 minutes. To one of the tubes, 10 μl each of CD73-CFS Mouse IgG2B, CD90-APC Mouse IgG2A, and Negative Marker Cocktail (CD45-PE Mouse IgG1, CD34-PE Mouse IgG1, CD11b-PE Mouse IgG2B, CD79A-PE Mouse IgG1, HLA-DR-PE Mouse IgG1), which were contained in Human Mesenchymal Stem Cell Verification Flow Kit (R&D Systems, FMC020), and further 5 μl of Brilliant Violet 421™ anti-human CD105 Antibody (BioLegend, 323219) were added, and allowed to react for 30 minutes at room temperature in the dark. Since the other tube was used as a negative control, the same amount of each isotype control antibody was added and allowed to react for 30 minutes at room temperature in the dark. The cells were washed once with 2 ml of Flow Cytometry Staining Buffer (1×), resuspended in 300 μl of Flow Cytometry Staining Buffer (1×), and analyzed by BD FACSAria (BD). In addition, whether CD31 was expressed in blister-derived cells and human BM-MSCs was analyzed by FACS analysis according to the following procedure. The cells were removed from the plate with Accutase-Solution (PromoCell, C-41310), and after washed with medium, 100,000 cells each were put into two tubes based on cell count results. The cells were washed once with 2% FBS-containing PBS and resuspended in 100 μl of 2% FBS-containing PBS. To block Fc receptors, 5 μl of Human TruStain FcX™ (BioLegend, 422301) was added and allowed to react on ice for 10 minutes. To one of the tubes, 5 μl of APC anti-human CD31 Antibody (BioLegend, 303116) (0.4 μg of protein) was added and allowed to react for 60 minutes on ice in the dark. Since the other tube was used as a negative control, 2 μl of APC Mouse IgG1, κ Isotype Ctrl Antibody (BioLegend, 400120) (0.4 μg of protein) was added and allowed to react for 60 minutes on ice in the dark. The cells were washed once with 2 ml of 2% FBS-containing PBS, resuspended in 300 μl of 2% FBS-containing PBS, and analyzed by BD FACSAria (BD).

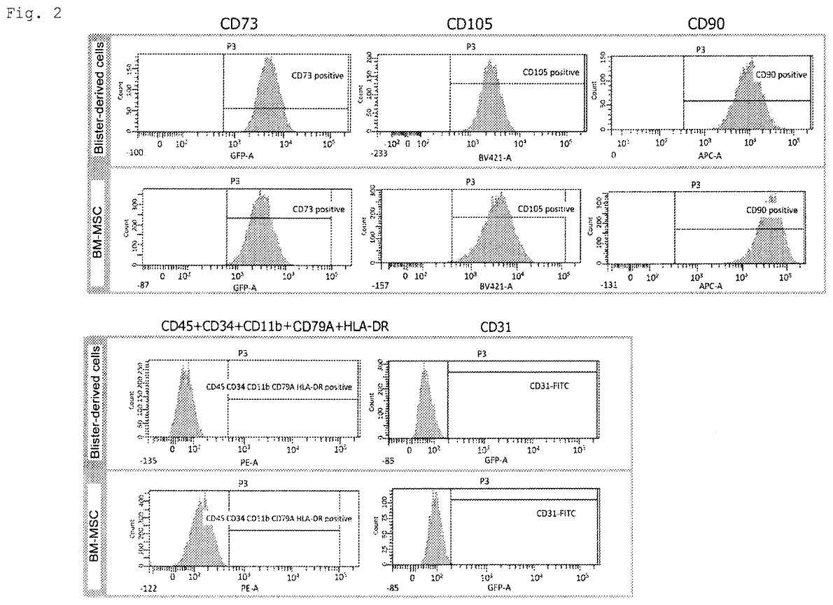

FACS analysis showed that both blister-derived cells and BM-MSCs were positive for CD73, CD105 and CD90 and negative for CD45, CD34, CD11b, CD79A, HLA-DR and CD31 (FIG. 2).

### b) Induction of Differentiation (Osteoblasts, Adipocytes and Chondrocytes)

The blister-derived cells obtained in the section 1 above and BM-MSCs were induced to differentiate into osteoblasts, adipocytes and chondrocytes under the following conditions.

Induction of Differentiation into Osteoblasts:

Cells were cultured in a medium containing 0.1 μM Dexamethasone, 0.2 mM Ascorbic acid 2-phosphate, 10 mM Glycerol 2-phosphate (all values were final concentrations) at 37° C. and 5% CO₂ for 3 weeks (the medium was changed twice a week) to induce differentiation into osteoblasts. The cells were stained by Alkaline phosphatase (ALP) staining using TRACP & ALP Assay Kit (Takara Bio Inc., MK301) according to the product manual.

Induction of Differentiation into Adipocytes:

Cells were cultured in a medium containing 1 μM Dexamethasone, 0.5 mM 3-Isobutyl-1-methylanxthine (IBMX), 10 μg/mL Insulin, and 100 μM Indomethacin (all values were final concentrations) at 37° C. and 5% CO₂ for 3 weeks (the medium was changed twice a week) to induce differentiation into adipocytes. The cells were stained by Oil Red O staining using a LipiD Assay kit (Cosmo Bio, AK09F) according to the product manual.

Induction of Differentiation into Chondrocytes:

A chondrogenic differentiation medium (incomplete medium) was prepared by mixing the components of Human Mesenchymal Stem Cell (hMSC) Chondrogenic Differentiation Medium Bullet Kit (tm) (Lonza, PT-3003) according to the instructions. Recombinant Human TGF-beta 3 Protein (R&D Systems, 243-B3) was added to the medium to a final concentration of 10 ng/ml to prepare a chondrogenic differentiation medium (complete medium) each time it was used. After the third passage cells were detached with Accutase-Solution PromoCell, C-41310) and washed with medium, 250,000 cells each were put into 15 ml polypropylene conical tubes based on cell count results. The cells were washed twice with the chondrogenic differentiation medium (incomplete medium), the supernatant was removed, and the cells were suspended in 500 μl of the chondrogenic differentiation medium (complete medium). The cells were centrifuged at 150 g for 5 minutes to form a cell pellet, and after the lid of the tube was unscrewed, the tube was placed in a CO₂ incubator (37° C., 5% CO₂), and then the medium (complete medium) was changed every 2 to 3 days. After 3 weeks, the pellet was removed and fixed with 4% paraformaldehyde, frozen sections were prepared, and chondrocyte-derived proteoglycans were stained by Alcian Blue staining. As a positive control, human bone marrow-derived mesenchymal stem cells were also subjected to the same differentiation-inducing operation.

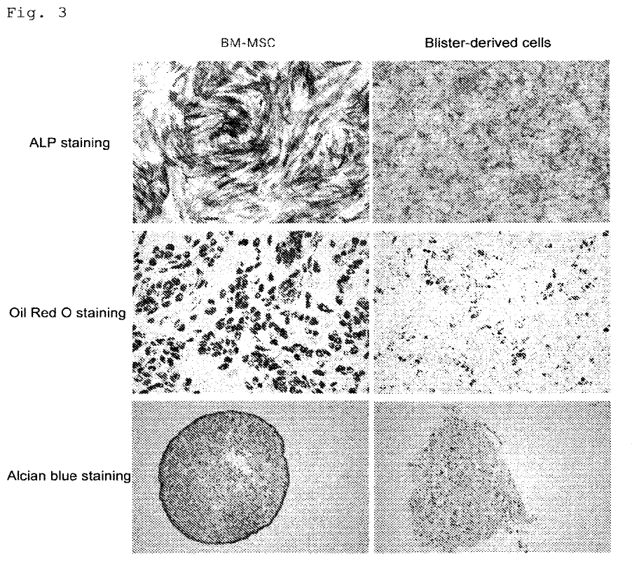

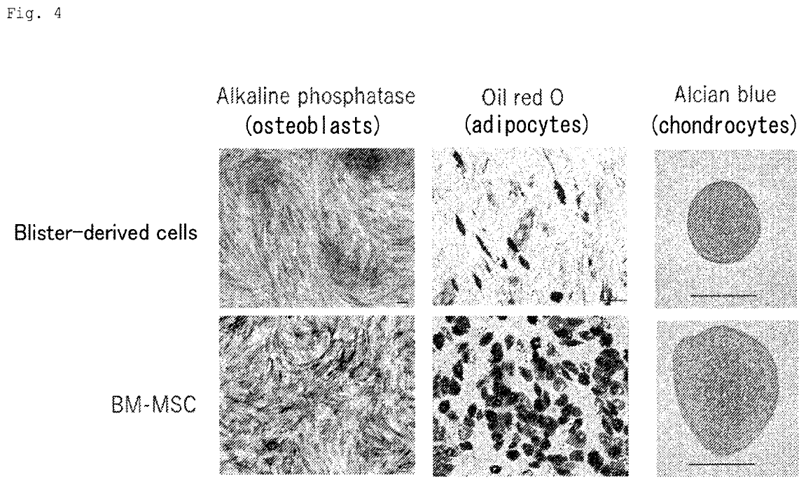

In the above differentiation induction experiments, BM-MSCs were positive in all of ALP staining, Oil Red O staining, and Alcian Blue staining. On the other hand, the blister-derived cells were positive in ALP staining (but staining intensity was lower than that of BM-MSCs), positive in Oil Red O staining (but staining intensity was lower than that of BM-MSCs), and negative in Alcian blue staining (FIG. 3). Such differentiation induction experiments were also performed on blister-derived cells obtained from a blister fluid collected from a different patient with dystrophic epidermolysis bullosa. The results are shown in FIG. 4. Similar to the results in FIG. 3, BM-MSCs were positive in all of ALP staining, Oil Red O staining, and Alcian Blue staining. On the other hand, the blister-derived cells of this patient with dystrophic epidermolysis bullosa were positive in ALP staining (but staining intensity was lower than that of BM-MSCs), positive in Oil Red O staining (but staining intensity was lower than that of BM-MSCs), and positive in Alcian blue staining (but staining intensity was lower than that of BM-MSCs).

### c) Evaluation of Ability to Express and Secrete Type VII Collagen

How much blister-derived cells expressed and secreted type VII collagen was examined by Western blotting. In this experiment, blister-derived cells were obtained from a patient who expressed type VII collagen, but whose expressed type VII collagen was thought to have little function due to amino acid mutation. First, human epidermal keratinocytes (hereinafter referred to as KC), human skin fibroblasts (hereinafter referred to as FB), human bone marrow-derived mesenchymal stem cells (hereinafter referred to as MSC), and blister-derived cells of an epidermolysis bullosa patient (hereinafter BFC; also referred to as blister fluid cells) were cultured in the media shown in Table 1 below.

TABLE 1

| Cells | Medium |
|---|---|
| KC: Human Epidermal | Humedia-KG2 (KURABO, KK- |
| Keratinocyte (NB)* | 2150S) |
| (KURABOU, KK-4009) | |
| *(NB means “New Born) | |
| FB: Normal Human Dermal | Fibroblast Growth Medium-2 |
| Fibroblasts - Adult (Lonza, | BulletKit (Lonza, CC-3132) |
| CC-2511) | |
| MSC: hMSC - Human | MSCGM Mesenchymal Stem Cell |
| Mesenchymal Stem Cells | Growth Medium (Lonza, PT- |
| (Lonza, PT-2501) | 3001) |
| BFC: blister-derived cells | Mixed medium of equal volume |
| (collected from blister | of Mesenchymal Stem Cell |
| fluid of epidermolysis | Growth Medium 2 (PromoCell, |
| bullosa patient) | C-28009) and MSCGM |
| | Mesenchymal Stem Cell Growth |
| | Medium (Lonza, PT-3001) |

When the cells reached 90-95% confluence, the cells were washed with D-PBS(−). Each medium (no supplement added) containing ascorbic acid (Nacalai Tesque, 13048-42, final concentration of 50 ug/ml) and a protease inhibitor cocktail (SIGMA, P1860-1ML, 1/400 dilution) was added to the cells, and the cells were cultured in a CO₂ incubator for 24 hours. After the culture, the medium was concentrated using a methanol-chloroform precipitation method. A cell lysate was also prepared from the cells using RIPA buffer (Nacalai Tesque, 08714-04). Each lysate was corrected based on protein concentration, and a sample for electrophoresis was prepared using LDS sample buffer and sample reducing agent (Invitrogen, NP0007 and NP0009, respectively). After electrophoresis with 3-8% NuPAGE gel (Invitrogen, EA0375BOX), the gel was transferred to a PVDF membrane (Millipore, IPVH07850), and the membrane was reacted with Anti-Col7 (Atlas, HPA042420) as a primary antibody and Anti-Rabbit IgG-HRP (GE healthcare, NA9340-1ML) as a secondary antibody. Then, the bands were detected using Chemi-lumi-one ultra (Nacalai Tesque, 11644-40) and Chemi DOC (BioRad, 17001402JA), and analyzed and quantified with Image Lab software (BioRad, 1709690). The western blotting was also performed on the concentrated medium in the same manner to quantify the concentration of type VII collagen.

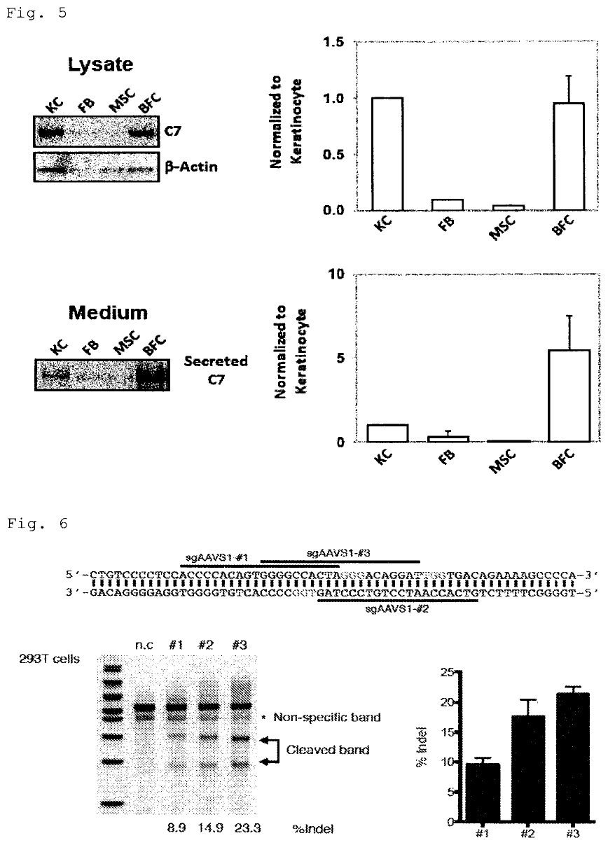

The results are shown in FIG. 5. As shown in FIG. 5 (top), the western blotting of the cell lysate revealed that the expression level of COL7A in blister-derived cells was higher than that of skin fibroblasts and bone marrow-derived mesenchymal stem cells, and at a level comparable to that of epidermal keratinocytes. In addition, as shown in FIG. 5 (bottom), the western blotting on the concentrate of the medium in which the cells were cultured revealed that the amount of COL7A secreted from the blister-derived cells was higher than that from any other cells. From these results, blister-derived cells were considered to be optimal cells to express type VII collagen by gene transfer.

### 3. Design of Genome Editing

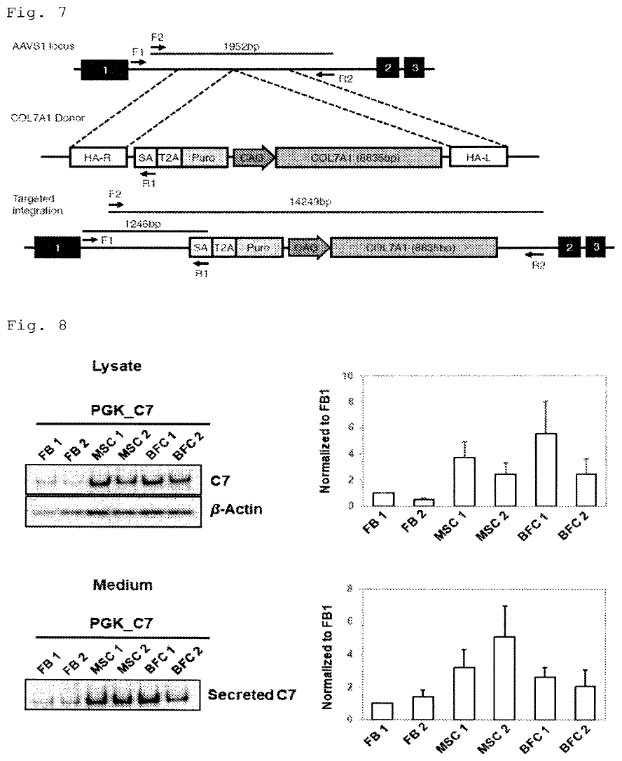

Three types of sgRNAs were prepared in order to select a site with good cleavage efficiency by CRISPR-Cas9 system in AAVS1 (Adeno-associated virus integration site 1) region in the human genome. The AAVS1 region is a safe region that is not easily affected by gene transfer (safe harbor). Since the CRISPR-Cas9 system recognized the base sequence of “NGG” and cleaved 3 bases upstream of the sequence, regions each having “GG” at the end were selected and sgRNAs each containing a target sequence of 20 bases upstream of “NGG” were designed (sgAAVS1-#1 to #3) (FIG. 6, top; Table 2).

TABLE 2

| sgRNA | Target sequence | SEQ ID NO. |
|---|---|---|
| sgAAVS1-#1 | ACCCCACAGTGGGGCCACTA | 3 |
| sgAAVS1-#2 | GTCACCAATCCTGTCCCTAG | 4 |
| sgAAVS1-#3 | GGGGCCACTAGGGACAGGAT | 5 |

An oligonucleotide consisting of a sequence of any one of SEQ ID NOs: 3 to 5 was annealed with its complementary strand and cloned into the Bbs1 site of eSpCas9 (1.1) (Addgene plasmid #71814) to prepare a plasmid that expressed Cas9 protein and sgRNA (eSpCas9(1.1)-sgAAVS1-#1, eSpCas9(1.1)-sgAAVS1-#2, or eSpCas9(1.1)-sgAAVS1-#3). This plasmid (2.5 μg) was introduced into HEK293 cells (human fetal kidney cell line) seeded in 6-well dishes by Lipofectamin 3000 (Thermo Fisher Scientific). Forty-eight hours after transfection, genomic DNA was extracted from the cells and the region containing the target site was amplified by PCR. The PCR amplified fragments were subjected to heat treatment to be single chains, and they were annealed by slow cooling and then treated with a mismatch site-specific endonuclease. The resulting product was fractionated by electrophoresis, the degree of insertion or deletion mutation introduced by the genome cleavage was measured from the density of the band, and the genome editing efficiency was calculated by the following formula (In the formula, “a” indicates the concentration of the band that was not digested, and “b” and “c” indicate the concentrations of the cleaved bands.).

Indel(%)=10033 (1−\sqrt{(1−fcut)}), f_{cut}=(b+c)/(a+b+c)

All sgRNAs of sgAAVS1-#1 to #3 produced a short DNA fragment different from the control, confirming that double-strand break occurred (FIG. 6, bottom). In the following experiments, sgAAVS1-#3, which had the highest cleavage efficiency, was used.

### 4. Introduction of COL7A1 Gene into Blister-Derived Cells

For introduction of a COL7A1 gene into the AAVS1 region, a plasmid expressing a COL7A1 gene under the control of a CAG promoter was designed (FIG. 7). COL7A1 cDNA was obtained from Flexi ORF sequence-verified clone (Promega, Madison, WI, USA). The COL7A1 cDNA was subcloned into the pENTR1A plasmid (Thermo Fisher Scientific, A10462) to prepare pENTR1A-COL7A1. COL7A1 cDNA was transferred from pENTR1A-COL7A1 to pAAVS1-P-CAG-DEST (Addgene plasmid #80490) by Gateway reaction using LR recombinase (Thermo Fisher Scientific) to obtain a donor plasmid pAAVS1-P-CAG-COL7A1.

The blister-derived cells obtained in the section 1 above were suspended in a special buffer for the Neon transfection system (Thermo Fisher Scientific), and mixed with the Cas9-sgRNA expression plasmid (eSpCas9(1.1)-sgAAVS1-#3) and the donor plasmid (pAAVS1-P-CAG-COL7A1) as follows.

TABLE 3

| Blister-derived cells | 1 × 10⁵ | cells |
|---|---|---|
| eSpCas9(1.1)-sgAAVS1-#3 | 0.5 | μg |
| pAAVS1-P-CAG-COL7A1 | 0.25 | μg |

Using the Neon transfection system, the plasmid was introduced into blister-derived cells by electroporation under the conditions of 1200 V, 20 ms, and 2 pulses, and the cells were seeded on a 6-well plate and cultured. The medium was a mixed medium of equal volume of Mesenchymal Stem Cell Growth Medium 2 (PromoCell, C-28009) and MSCGM Mesenchymal Stem Cell Growth Medium (Lonza, PT-3001). Forty-eight hours after transfection, puromycin was added to a final concentration of 0.5 μg/mL, and the cells were cultured for about 2 weeks. The selected cells were treated as described in “5. Transplantation of genetically modified blister-derived cells into mice”.

We also constructed a donor plasmid that expressed a COL7A1 gene under the control of a PGK promoter, and introduced this plasmid into various cells, including blister-derived cells. Then, the expression level and secretion level of COL7A1 in the modified cells were evaluated by Western blotting in the same manner as in “c) Evaluation of ability to express and secrete type VII collagen” in “2. Characterization of blister-derived cells” above. In this experiment, blister-derived cells were obtained from a patient who did not express type VII collagen. The results are shown in FIG. 8. As shown in FIG. 8 (top), more type VII collagen was detected in the blister-derived cell lysate than in the fibroblast lysate. Similarly, as shown in FIG. 8 (bottom), more type VII collagen was detected in the medium in which blister-derived cells were cultured than in the medium in which fibroblasts were cultured. From these results, it was found that when a COL7A gene was introduced into cells with CRISPR-Cas9, blister-derived cells expressed and secreted more type VII collagen than fibroblasts. (It was confirmed by immunostaining and Western blotting of culture supernatant that cells expressing and secreting type VII collagen were obtained even when blister-derived cells were genetically modified with a COL7A1 gene donor plasmid using an EF1α promoter.)

### 5. Transplantation of Genetically Modified Blister-Derived Cells into Mice

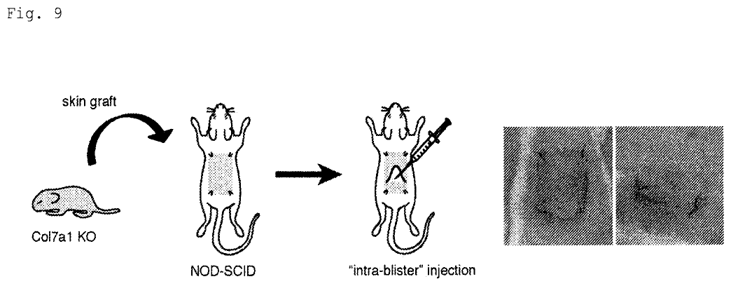

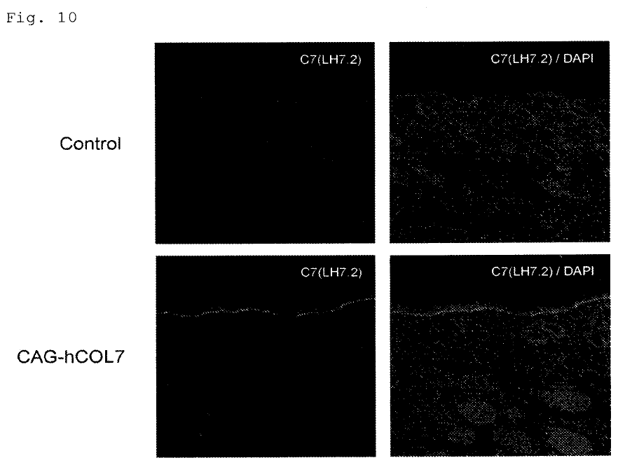

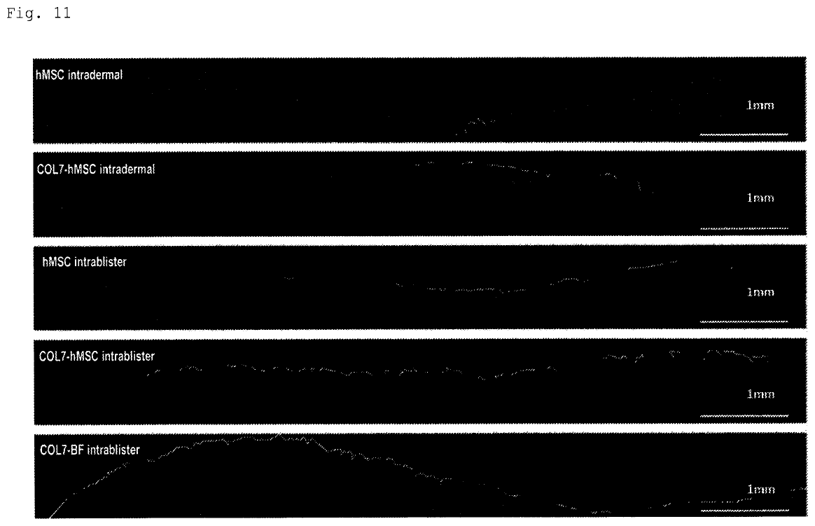

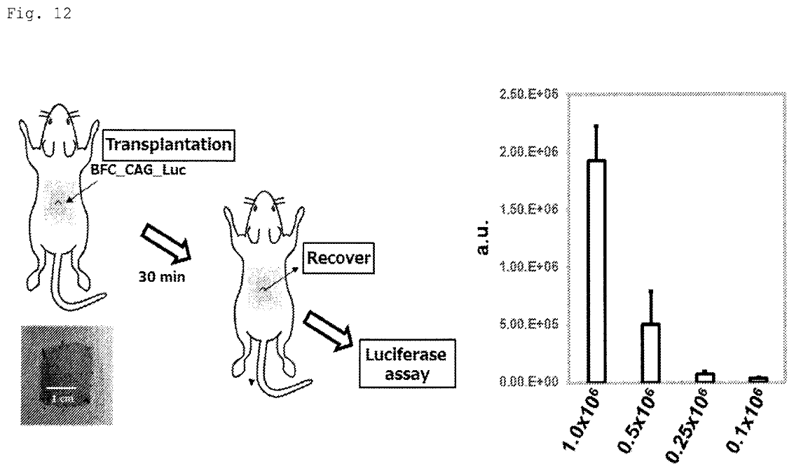

The full-thickness skin of a neonatal Col7A1 gene knockout mouse (Col7a1-/-) showing blistering was excised and transplanted to the back of an immunodeficient mouse (NOD-SCID). Immediately after the transplantation, the skin surface was pinched and rubbed to form a blister, and 1.0×10⁶ genetically modified blister-derived cells prepared in the section 4 above were immediately injected into the space under the epidermis (into the blister) (FIG. 9) and the injection site was sealed with a film dressing. (To a control mouse, 1.0×10 6 non-genetically modified blister-derived cells were injected.) After 4 weeks, the skin was collected and immunostained with an anti-type VII collagen antibody (clone LH7.2; Sigma Aldrich, C6805) to confirm the deposition of type VII collagen on the basement membrane. Deposition of type VII collagen was observed in the vicinity of the basement membrane in the mouse which were injected with the genetically modified blister-derived cells by intrablister injection (FIG. 10).

### 6. Deposition of Type VII Collagen in Genetically Modified Cells-Transplanted Mice

We compared type VII collagen deposition between blister-derived cells and mesenchymal stem cells in epidermolysis bullosa model mice. First, the full-thickness skin of a neonatal Col7A1 gene knockout mouse was transplanted to the back of an immunodeficient mouse (NOD-SCID). Then, 1.0×10⁶ of the following cells were injected into the dermis (intradermally) or into the space under the epidermis (into the blister).
hMSC: Non-genetically modified human bone marrow-derived mesenchymal stem cells
COL7-hMSC: Genetically modified (type VII collagen-overexpressing) human bone marrow-derived mesenchymal stem cells, which were prepared by introducing a COL7A1 gene into human bone marrow-derived mesenchymal stem cells (hMSC) [purchased from Lonza (Basel, Switzerland)] by the same method as described in “4. Introduction of COL7A1 gene into blister-derived cells”
COL7-BF: Genetically modified blister-derived cells prepared in “4. Introduction of COL7A1 gene into blister-derived cells”

After 4 weeks, the skin was collected and immunostained with an anti-type VII collagen antibody (clone LH7.2; Sigma Aldrich, C6805), and photographs of stained images were taken. Then, using an image analysis software, a plurality of photographs were superimposed so that the stained portion of type VII collagen was correctly matched to provide a merged image.

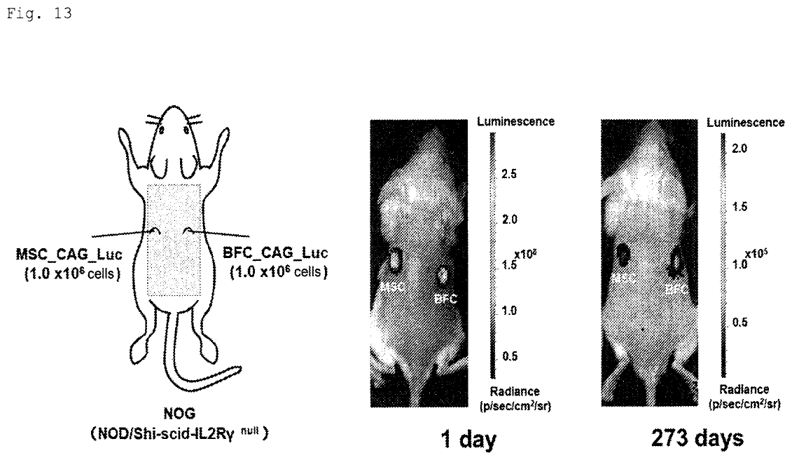

The results are shown in FIG. 11. As shown by the comparison of “hMSC” with “hMSC intrablister” and that of “COLT-hMSC intradermal” with “COL7-hMSC intrablister” in FIG. 11, more type VII collagen was detected around the basement membrane when the mesenchymal stem cells were injected into the blister than when the cells were injected intradermally. Also, as shown by the comparison of “COL7-hMSC intrablister” with “COLT-BF intrablister” in FIG. 11, much more type VII collagen was detected around the basement membrane when the COL7A1 gene-introduced blister-derived cells were injected into the blister than when the COL7A1 gene-introduced mesenchymal stem cells were injected into the blister. From these results, it was found that intrablister injection of COL7A1 gene-introduced cells resulted in better deposition of type VII collagen in the skin than intradermal injection. It was also found that more type VII collagen could be deposited around the basement membrane when blister-derived cells were used.

### 7. Number of Blister-Derived Cells to be Transplanted into Blister

It was investigated how many blister-derived cells should be transplanted into a blister to obtain the necessary effect. As shown in FIG. 12 (left), first, the full-thickness skin of a neonatal Col7A1 gene knockout mouse (Col7a1-/-) showing blistering was excised and transplanted to the back of an immunodeficient mouse (NOG). Immediately after the transplantation, the skin surface was pinched and rubbed to form a blister having about 1 cm of diameter. Then, 1.0×10⁶, 0.5×10⁶, 0.25×10⁶ or 0.1×10⁶ blister-derived cells genetically modified with a gene donor plasmid of firefly luciferase regulated by a CAG promoter were immediately injected into the space under the epidermis (into the blister) of the mouse. After 30 minutes, the blister fluid was collected and the firefly luciferase level was measured using a Luciferase assay system (Promega, E2510) to determine the number of remaining cells in the blister fluid that were not grafted to the dermis.

The results are shown in FIG. 12 (right). As shown in the figure, there was almost no difference in signal intensity in the group in which 0.25×10⁶ cells or 0.1×10⁶ cells were transplanted. On the other hand, in the group in which 0.5×10⁶ cells or 1.0×10⁶ cells were transplanted, the signal increased in proportion to the increase of cell number. These results suggested that a sufficient number of cells would graft to the basement membrane to obtain the therapeutic effect when 0.5×10⁶ or more cells per blister with a diameter of 1 cm (=about 0.8 cm²) (=0.6×10⁶ cells/cm² or more) are injected into a blister.

### 8. Long-Term Grafting Potential of Transplanted Cells

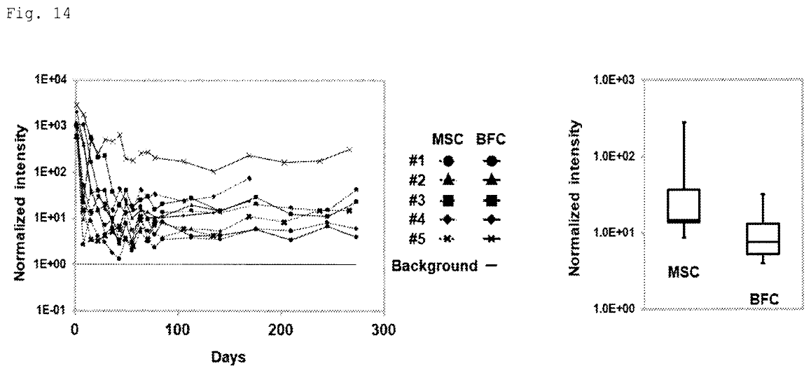

It was investigated how long blister-derived cells administered intradermally to a mouse survived at the administration site. First, as shown on FIG. 13 (left), 1×10⁶ bone marrow-derived mesenchymal stem cells or blister-derived cells genetically modified with a gene donor plasmid of firefly luciferase regulated by a CAG promoter were administered by intradermal injection to the left and right sides of the back of an immunodeficient mouse (NOG). Periodically, 100 μl of 30 mg/ml luciferin (Promega, P1042) was administered by intraperitoneal injection, and the luminescence was observed with IVIS Lumina II Imaging System (Caliper).

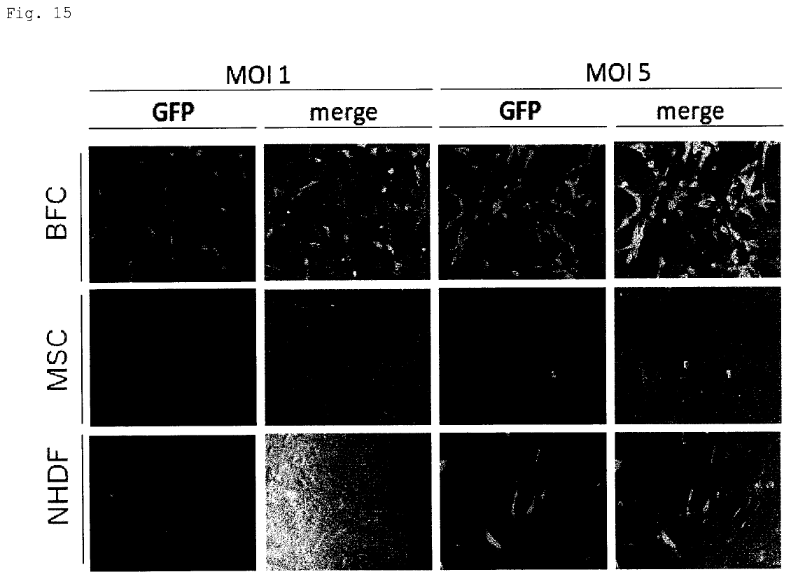

The results are shown in FIG. 13 (right) and FIG. 14. As shown in FIG. 13 (right) and FIG. 14 (left), the luminescence signal intensity gradually decreased during the first month in both blister-derived cells and bone marrow-derived mesenchymal stem cells, and then became almost constant and maintained for more than 9 months. As shown in FIG. 14 (right), when the average value of the signal intensity of individual mice after one month was analyzed, no difference was observed in the luminescence signal intensity between blister-derived cells and bone marrow-derived mesenchymal stem cells. From these results, it was found that the blister-derived cells had a high grafting potential comparable to that of bone marrow-derived mesenchymal stem cells.

### 9. Infection Efficiency of Lentivirus to Blister-Derived Cells

The infection efficiency of lentivirus to blister-derived cells was analyzed. The cells used in this experiment and the medium used to culture the cells are shown in Table 4 below.

TABLE 4

| Cells | Medium |
|---|---|
| BFC: blister-derived cells | Mixed medium of equal volume |
| (collected from blister | of Mesenchymal Stem Cell |
| fluid of epidermolysis | Growth Medium 2 ([PromoCell |
| bullosa patient) | (Heidelberg, Germany), C- |
| | 28009] and MSCGM Mesenchymal |
| | Stem Cell Growth Medium |
| | [Lonza (Basel, Switzerland), |
| | PT-3001] |
| MSC: human bone marrow- | Mesenchymal Stem Cell Growth |
| derived mesenchymal stem | Medium [Lonza (Basel, |
| cells [Lonza (Basel, | Switzerland), PT-3001] |
| Switzerland), PT-2501] | |
| NHDF: normal human adult | FGM ™-2 Fibroblast Growth |
| skin fibroblasts [Lonza | Medium-2 BulletKit ™ [Lonza |
| (Basel, Switzerland), CC- | (Basel, Switzerland), CC- |
| 2511] | 3132)] |

Preparation of RetroNectin-coated plate: RetroNectin [Takara Bio Inc. (Shiga, Japan), T100B] was diluted to 40 μg/mL with PBS (Dulbecco's phosphate-buffered saline (Ca, Mg-free)) [Nacalai Tesque (Kyoto, Japan), 14249-95] and added to a 96-well plate with no surface treatment [Corning (Tokyo, Japan), 3370] at 100 μL/well. The plate was then left overnight at 4° C. Before the plate was used, the RetroNectin solution was removed, the plate was washed twice with PBS, and the following operations were performed.

Cell seeding and lentivirus infection: The cells were detached from the plate with Accutase-Solution [PromoCell (Heidelberg, Germany), C-41310] for blister-derived cells, Trypsin/EDTA for Mesenchymal Stem Cells [Lonza (Basel, Switzerland), CC-3232] for human bone marrow-derived mesenchymal stem cells, and Trypsin/EDTA Solution [Lonza (Basel, Switzerland), CC-5012] for normal human adult skin fibroblasts, and collected by using the cell culture medium for respective cells. The number of collected cells was counted and the cells were seeded on the RetroNectin-coated 96-well plate at 2500 cells/well. After that, a lentivirus having a GFP gene: pLenti-C-mGFP [ORIGENE (Rockville, USA), PS100071] was added to each well at MOT 1 or MOT 5, and the cells were cultured in a CO₂ incubator for 72 hours.

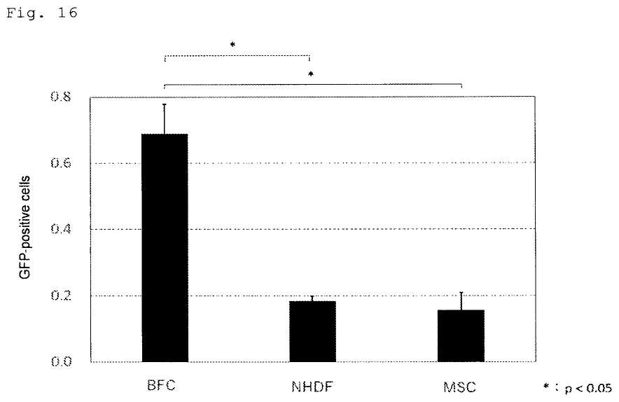

Detection of GFP-positive cells: The GFP-positive cell ratio was detected with a fluorescence microscope [Keyence (Tokyo, Japan), BZ-X710]. The results are shown in FIG. 15.

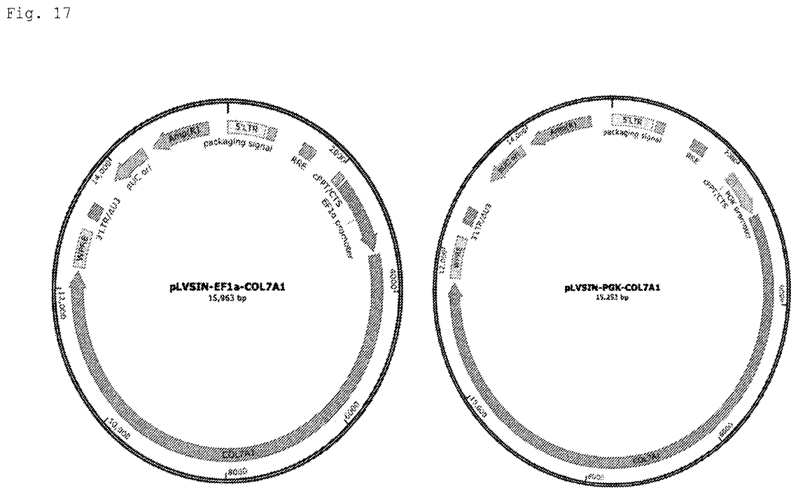

Also, GFP-positive cells after infection with MOT 1 were quantified by the following procedure for each cell type. First, the ratio of the number of GFP-positive cells to the total number of cells in a visual field was defined as the GFP-positive cell ratio. This measurement was repeated three times and statistical processing was performed (*: P<0.05, Dunnett's test). The results are shown in FIG. 16.

As shown in FIG. 15, it was found that the blister-derived cells showed a higher GFP-positive cell ratio and a higher GFP fluorescence intensity than the mesenchymal stem cells and fibroblasts. Further, as shown in FIG. 16, the GFP-positive cell ratio of the blister-derived cells was found to be significantly higher than that of the mesenchymal stem cells or fibroblasts. These results suggest that the efficiency of lentiviral gene delivery to blister-derived cells is higher than those to mesenchymal stem cells and fibroblasts.

### 10. Preparation of Lentiviral Vector Plasmid Having Type VII Collagen Gene

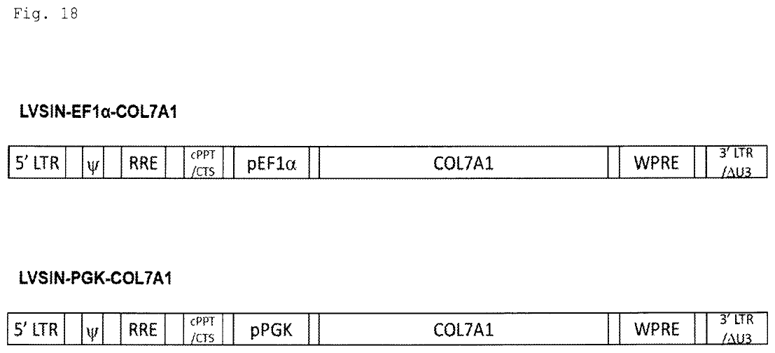

A lentiviral vector plasmid containing an EF1α promoter and a COL7A1 gene in an expression cassette as shown in FIG. 17 (left) was prepared. First, from the Flexi ORF sequence-verified clone (Promega) containing COL7A1 cDNA, the COL7A1 cDNA was cut out by treatment with SpeI and XbaI, which produced a paired sequence, and ligated to pLVSIN-EF1α Puro (Takara Bio) treated with XbaI to produce pLVSIN-EF1α-C7 Puro. The resulting product was further treated with NotI and MluI to remove the PGK-Puro cassette to provide pLVSIN-EF1α-COL7A1.

Also, a lentiviral vector plasmid containing a PGK promoter and a COL7A1 gene in an expression cassette as shown in FIG. 17 (right) was prepared. The EF1α promoter region was cut out from pLVSIN-EF1α-Col7A1 by treatment with ClaI and SwaI, and the PGK promoter region (501 bp) was amplified by PCR using AAVS1 hPGK-PuroR-pA donor plasmid (Addgene #22072) as a template, and the PGK promoter region was integrated by Gibson assembly to provide pLVSIN-PGK-COL7A1. The PGK promoter was amplified with KOD One (Toyobo). For the Gibson assembly, NEBuilder HiFi DNA Assembly Kit (New England Biolabs) was used.

### 11. Production of Lentiviral Vector Having Type VII Collagen Gene

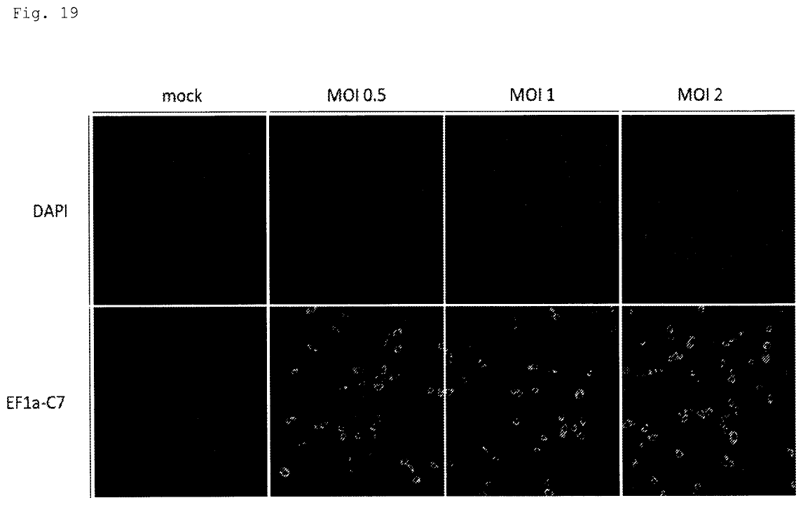

A lentiviral vector having a COL7A1 gene as shown in FIG. 18 was prepared.

Transfection using lentiviral plasmids: The plasmids shown in FIG. 17 were used as lentiviral vector plasmids. The packaging plasmid used was Lentiviral High Titer Packaging Mix [Takara Bio (Shiga, Japan), 6194]. First, Lenti-X 293T cells [Takara Bio Inc. (Shiga, Japan), 632180] (cell line for lentivirus packaging) were placed in a 100 mm dish [Corning (New York, USA), 353003] at 5000000 cells/dish and cultured overnight in a CO₂ incubator. The medium for Lenti-X 293T cells was DMEM [Nacalai Tesque (Kyoto, Japan), 08457-55] containing 10% FBS and penicillin/streptomycin (added to final concentrations of 100 unit/mL and 100 μg/mL, respectively). The vector plasmid and the packaging plasmid were then transfected using polyethylenimine. The transfected cells were cultured overnight in a CO₂ incubator and then the medium was changed. The transfection reagents used were OPTI-MEM [Thermo Fisher Science (Tokyo, Japan), 31985062] and PEI-MAX [Polysciences (Warrington, USA), 24765-100]. The PEI-MAX recommended protocol was used as a transfection protocol. The recommended protocol of Lentiviral High Titer Packaging Mix was used for the mixing ratio of the vector plasmid and packaging plasmid.

Collection and purification of lentiviral vector: The culture supernatant of Lenti-X 293T cells 72 hours after transfection was collected and coarsely centrifuged at 300 g for 5 min to remove cell debris. The supernatant was filtered using a 0.45 μm filter [Merck (Tokyo, Japan), SLHVR33RS] to further remove cell debris. The filtered supernatant was then centrifuged (6000 g, 4° C., 20 hr) to pellet the lentiviral vector and the pellet was resuspended in 1.5 mL of PBS. Next, in an ultracentrifuge tube [Beckman Coulter (Tokyo, Japan), 344058], 55% sucrose/PBS solution (1 mL), 20% sucrose/PBS solution (2.5 mL), and the lentiviral vector solution (1.5 mL) were layered and ultracentrifuged (41000 rpm, 4° C., 2 hr). The ultracentrifuge and the rotor used were [Beckman Coulter (Tokyo, Japan), L-90K] and [Beckman Coulter (Tokyo, Japan), SW55Ti]. After ultracentrifugation, the lentiviral vector layer appearing between 55% sucrose/PBS solution and 20% sucrose/PBS solution was collected and diluted to 1 mL with PBS. Next, 20% sucrose/PBS solution (4 mL) and the lentiviral vector solution (1 mL) were layered in an ultracentrifugation tube, and ultracentrifuged again (41000 rpm, 4° C., 2 hr). The precipitated lentiviral vector pellet was well suspended in 400 μL of DMEM to obtain a lentiviral vector solution.

The lentiviral vector titer was determined as follows.

Preparation of RetroNectin-coated plate: RetroNectin [Takara Bio Inc. (Shiga, Japan), T100B] was diluted with PBS to 100 μg/mL and added to a flat-bottom 48-well plate with no surface treatment [IWAKI (Tokyo, Japan), 1830-048] at 100 μL/well. The plate was then left overnight at 4° C. Before the plate was used, the RetroNectin solution was removed, and the plate was blocked with 2% FBS-containing PBS at room temperature for 30 minutes, followed by the following procedures.

Cell seeding and lentiviral infection: To the RetroNectin-coated 48-well plate, the LVSIN-EF1α-COL7A1 lentiviral vector or LVSIN-PGK-COL7A1 lentiviral vector (see FIG. 18) was added with a certain volume of virus solution in each well and centrifuged at 2000 g and 32° C. for 2 hours. Then, the virus solution was removed and the plate was washed once with PBS. Next, the blister-derived cells were detached from the plate using Accutase-Solution and collected by using a medium. The collected cells were counted and seeded on the RetroNectin-coated 48-well plate at 12500 cells/well. Cells 14 days after infection were detached from the plate with Accutase-Solution and collected by using a medium. Then, genomic DNA was extracted with Maxwell® RSC Instrument [Promega (Tokyo, Japan), AS4500] and Maxwell® RSC Blood DNA Kit [Promega (Tokyo, Japan), AS1400]. By using Lenti-X™ Provirus Quantitation Kit [Takara Bio Inc. (Shiga, Japan), 631239], Vector Copy Number (VCN) was calculated from the collected genomic DNA. The virus titer (TU/mL, TU: Transduction Unit) was calculated from the VCN and the volume of virus solution at the time of virus infection. The calculation formula is as follows.

$$Virus titer ( TU / mL ) = \frac{\begin{matrix}VCN ( copies / cell ) \times Number of cells at the \\ time of infection ( cells )\end{matrix}}{\begin{matrix}Volume of virus solution at the \\ time of virus infection ( μL )\end{matrix}} \times \frac{1000 μL}{1 mL}$$

Based on the virus titer thus obtained, the volume of virus solution required for setting the Multiplicity Of Infection (MOI) in the lentivirus infection experiment was calculated.

### 12. Analysis of Type VII Collagen Gene Transfer Efficiency by Immunostaining

We analyzed the efficiency of type VII collagen gene transfer by lentiviral vectors to blister-derived cells.

Preparation of RetroNectin-coated plate: A plate was coated with RetroNectin in the same manner as in “11. Production of lentiviral vector having type VII collagen gene”.

Cell seeding and lentiviral infection: To the RetroNectin-coated 48-well plate, the LVSIN-EF1α-COL7A1 lentiviral vector or LVSIN-PGK-COL7A1 lentiviral vector (see FIG. 18) was added at MOI 0.5, MOI 1, or MOI 2 in each well and centrifuged at 2000 g and 32° C. for 2 hours. Then, the virus solution was removed and the plate was washed once with PBS. Next, the blister-derived cells were detached from the plate using Accutase-Solution and collected by using a medium. The collected cells were counted and seeded on the RetroNectin-coated 48-well plate at 12500 cells/well.

Immunostaining: The blister-derived cells 14 days after lentivirus infection were detached from the plate with Accutase-Solution and collected by using a medium. The collected cells were counted, seeded on a CC2-coated chamber slide [Thermo Fisher Science (Tokyo, Japan), 154852] at 50000 cells/well, and cultured in a CO₂ incubator. After 24 hours, immunostaining was performed by using an anti-type VII collagen antibody (clone LH7.2) [Sigma Aldrich (Tokyo, Japan), C6805] and Alexa488 [Thermo Fisher Science (Tokyo, Japan), A-11001). Then, the expression of type VII collagen was analyzed with a confocal fluorescence microscope [Nikon (Tokyo, Japan), Nikon AIR HD25].

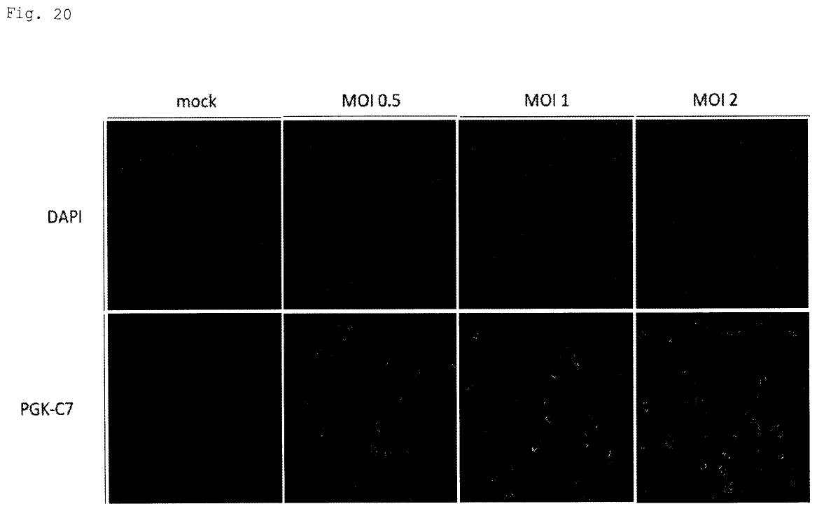

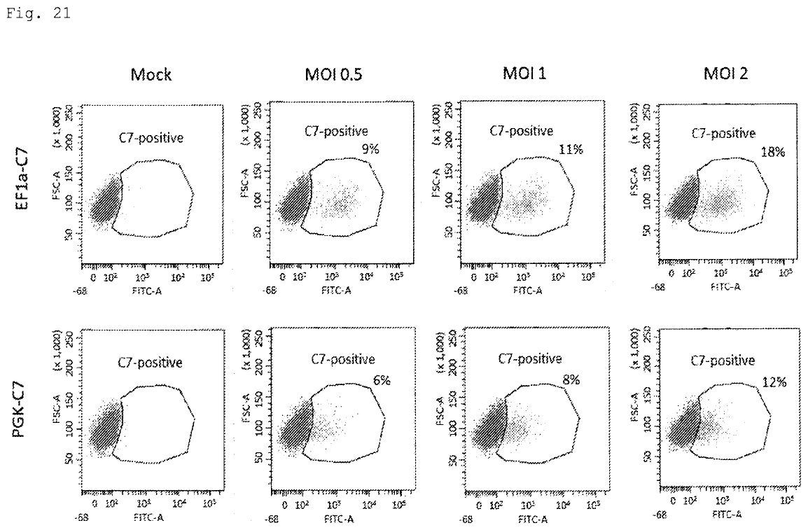

Results are shown in FIGS. 19 and 20. Infection of cells with the lentiviral vector having either EF1α promoter or PGK promoter increased the expression of type VII collagen in an MOI-dependent manner. The highest ratio of type VII collagen-positive cells was observed in the cells infected at MOI 2 with any of the vectors, approximately 30% with the LVSIN-EF1α-COL7A1 lentiviral vector and approximately 16% with the LVSIN-PGK-COL7A1 lentiviral vector. Also, in general, blister-derived cells infected with the LVSIN-EF1α-COL7A1 lentiviral vector showed a higher ratio of type VII collagen-positive cells and stronger staining intensity than the blister-derived cells infected with the LVSIN-PGK-COL7A1 lentiviral vector.

### 13. Analysis of Type VII Collagen Gene Transfer Efficiency by Flow Cytometry (FACS)

We analyzed the efficiency of type VII collagen gene transfer by lentiviral vectors to blister-derived cells.

Preparation of lentivirus-infected cells: The cells were prepared in the same manner as described in “12. Analysis of type VII collagen gene transfer efficiency by immunostaining”.

FACS analysis: Blister-derived cells 14 days after lentivirus infection were detached from the plate with Accutase-Solution and collected by using a medium. The number of collected cells were measured and the cells were divided to 300000 cells/sample and permeabilized by using eBioscience™ Permeabilization Buffer [Thermo Fisher Science (Tokyo, Japan), 00-8333-56]. Then, immunostaining was performed by using an anti-type VII collagen antibody (clone LH7.2) [Sigma Aldrich (Tokyo, Japan), C6805] and Alexa488 [Thermo Fisher Science (Tokyo, Japan), A-11001), and the expression of type VII collagen was analyzed by a flow cytometer (BD FACSCanto™ II) [Nippon Becton Dickinson (Tokyo, Japan)].

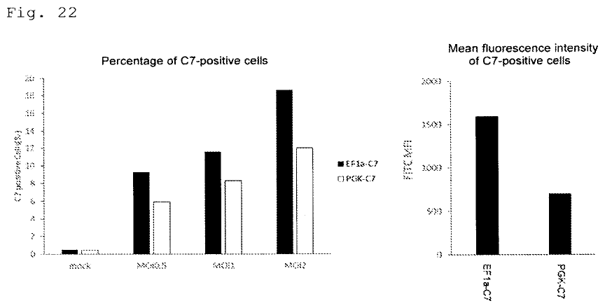

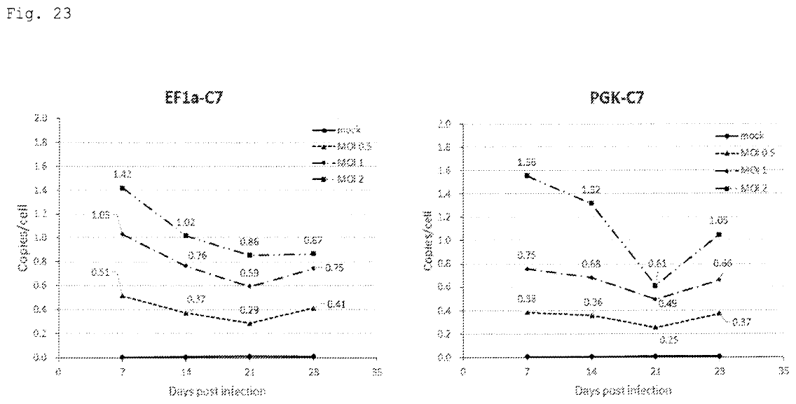

The results are shown in FIG. 21. The percentage of the cells contained in the encircled region in each FACS data is shown in FIG. 22 (left) as the percentage of type VII collagen-positive cells. Further, the Mean Fluorescence Intensity (MFI) within the encircled region is shown in FIG. 22 (right) as the mean fluorescence intensity of type VII collagen-positive cells. As shown in FIG. 21 and FIG. 22 (left), the percentage of type VII collagen-positive cells increased in an MOI-dependent manner. The highest percentage of type VII collagen-positive cells was observed with MOI 2 infection, approximately 18% with EF1α-COL7A1 lentivirus and approximately 12% with PGK-COL7A1 lentivirus. Also, as shown in FIG. 22 (right), the MFI of EF1α-COL7A1 lentivirus-infected cells was about 2.2 times higher than that of PGK-COL7A1 lentivirus-infected cells. These results suggest that the EF1α promoter induces expression of a higher amount of COL7A1 than the PGK promoter in the blister-derived cells transduced with a lentiviral vector.

Also, as a preliminary study, blister-derived cells, human bone marrow-derived mesenchymal stem cells, and normal human adult skin fibroblasts were infected with the EF1α-COL7A1 lentiviral vector or PGK-COL7A1 lentiviral vector, and immunostaining and flow cytometry (FACS) were performed in the same manner as above to measure the percentage of type VII collagen-positive cells. The blister-derived cells showed a higher percentage of type VII collagen-positive cells than the human bone marrow-derived mesenchymal stem cells and normal human adult skin fibroblasts.

### 14. Vector Copy Number (VCN) of Lentivirus-Infected Blister-Derived Cells

VCN of blister-derived cells infected with the lentiviral vector having the type VII collagen gene was analyzed. First, the blister-derived cells were infected with a lentiviral vector in the same manner as in “12. Analysis of type VII collagen gene transfer efficiency by immunostaining”. The cells 7, 14, 21, and 28 days after infection were detached from the plate with Accutase-Solution and collected by using a medium. Next, from the collected cells, genomic DNA was extracted by using Maxwell® RSC Instrument [Promega (Tokyo, Japan), AS4500] and Maxwell® RSC Blood DNA Kit [Promega (Tokyo, Japan), AS1400]. VCN was calculated from the collected genomic DNA by using the Lenti-X™ Provirus Quantitation Kit [Takara Bio Inc. (Shiga, Japan), 631239].

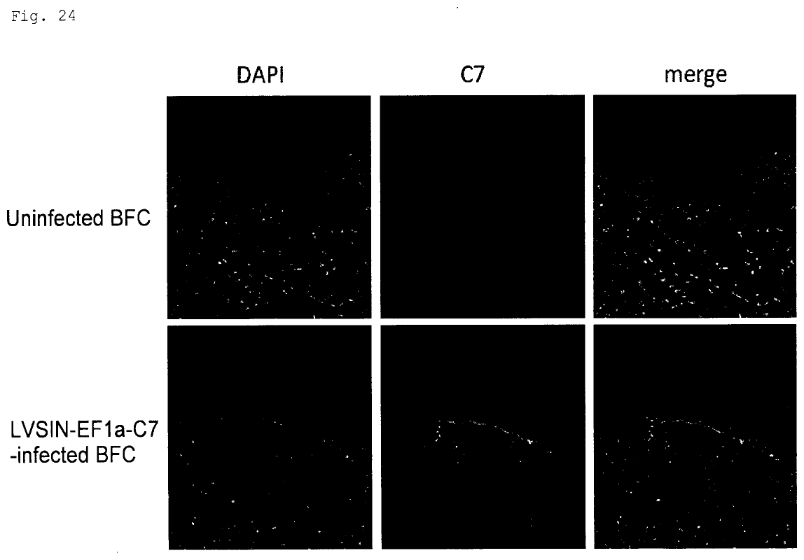

The results are shown in FIG. 23. As shown in the figure, VCN increased in an MOI-dependent manner. Also, VCN decreased as the days passed after the infection, but stabilized after the day 21. These results suggest that the type VII collagen gene inserted into the genome of blister-derived cells by a lentiviral vector can be maintained in the genome for a long period of time.

### 15. Deposition of Type VII Collagen in Mice to which Blister-Derived Cells Genetically Modified with Lentiviral Vector were transplanted

We examined whether the blister-derived cells transduced with the lentiviral vector having a type VII collagen gene supplied type VII collagen to the basement membrane of epidermolysis bullosa model mice. First, the full-thickness skin of a neonatal Col7A1 gene knockout mouse was transplanted to the back of an immunodeficient mouse. Immediately after the transplantation, the skin surface was pinched and rubbed to forma blister. Then, 1.0×10⁶ blister-derived cells which were prepared in “12. Analysis of type VII collagen gene transfer efficiency by immunostaining” and infected with the LVSIN-EF1α-COL7A1 lentiviral vector at MOT 5 were immediately injected into the space under the epidermis (into the blister). To the mouse in the control group, 1.0×10⁶ blister-derived cells not infected with the lentiviral vector were injected into the blister. One month later, the skin was collected and immunostained with an anti-type VII collagen antibody (clone LH7.2; Sigma Aldrich, C6805) to examine the deposition of type VII collagen at the basement membrane.

The results are shown in FIG. 24. As shown in the figure, deposition of type VII collagen was observed around the basement membrane of the mouse which were injected with the blister-derived cells infected with the LVSIN-EF1α-COL7A1 lentiviral vector by intrablister injection. On the other hand, no type VII collagen deposition was observed in the mouse which were injected with the blister-derived cells not infected with the lentiviral vector by intrablister injection. From these results, it was suggested that the blister-derived cells in which type VII collagen was introduced by the lentiviral vector grafted in the vicinity of the injection site after they were administered into the blister, and were able to supply type VII collagen to the adhesion site between the dermis and basement membrane.

From the above results, COL7A1 gene-delivered blister-derived cells are expected to exhibit higher therapeutic effects than bone marrow-derived mesenchymal stem cells and fibroblasts for gene therapy for dystrophic epidermolysis bullosa.

1-15. (canceled)

16. A method of treating dystrophic epidermolysis bullosa, comprising the step of administering to a patient with dystrophic epidermolysis bullosa a cell derived from a blister of the patient, wherein the cell is genetically modified to produce type VII collagen.

17. The method of claim 16, wherein the cell is genetically modified by introducing a COL7A1 gene to the cell.

18. The method of claim 17, wherein the COL7A1 gene comprises a nucleic acid sequence having 90% or more sequence identity with the nucleic acid sequence of SEQ ID NO: 1, or a nucleic acid sequence that encodes an amino acid sequence having 90% or more sequence identity with the amino acid sequence of SEQ ID NO: 2.

19. The method of claim 16, wherein the cell has one or more characteristics selected from the group consisting of following 1)-6):
1) adherent to a solid surface;
2) positive for one or more surface markers selected from the group consisting of CD73, CD105 and CD90;
3) negative for one or more surface markers selected from the group consisting of CD45, CD34, CD11b, CD79A, HLA-DR and CD31;
4) incapable of differentiating into an osteoblast, or less capable of differentiating into an osteoblast than a bone marrow-derived mesenchymal stem cell;
5) incapable of differentiating into an adipocyte, or less capable of differentiating into an adipocyte than a bone marrow-derived mesenchymal stem cell; and
6) incapable of differentiating into a chondrocyte, or less capable of differentiating into a chondrocyte than a bone marrow-derived mesenchymal stem cell.

20. The method of claim 16, wherein the cell is derived from a fluid in the blister of the patient.

21. The method of claim 16, wherein the cell is administered into another blister of the patient.

22. A method of treating dystrophic epidermolysis bullosa, comprising the steps of:
obtaining a cell from a fluid in a blister of a patient with dystrophic epidermolysis bullosa;
genetically modifying the cell to produce type VII collagen; and
administering the cell to the patient.

23. The method of claim 22, further comprising the step of administering the cell into another blister of the patient.

24. A method of producing a cell, comprising the step of culturing a content in a blister of a patient with dystrophic epidermolysis bullosa on a solid surface.

25. A cell produced by the method of claim 24.

26. The cell of claim 25, wherein the cell has one or more characteristics selected from the group consisting of following 1)-6):
1) adherent to a solid surface;
2) positive for one or more surface markers selected from the group consisting of CD73, CD105 and CD90;
3) negative for one or more surface markers selected from the group consisting of CD45, CD34, CD11b, CD79A, HLA-DR and CD31;
4) incapable of differentiating into an osteoblast, or less capable of differentiating into an osteoblast than a bone marrow-derived mesenchymal stem cell;
5) incapable of differentiating into an adipocyte, or less capable of differentiating into an adipocyte than a bone marrow-derived mesenchymal stem cell; and
6) incapable of differentiating into a chondrocyte, or less capable of differentiating into a chondrocyte than a bone marrow-derived mesenchymal stem cell.

27. A method of producing a cell, comprising the steps of:
culturing a content in a blister of a patient with dystrophic epidermolysis bullosa to obtain a cell; and
genetically modifying the cell to produce type VII collagen.

28. A method of producing a cell, comprising the steps of:
inoculating a content in a blister of a patient with dystrophic epidermolysis bullosa in a medium without treating the content with an enzyme;
incubating the medium to grow a cell at a bottom of a container containing the medium; and
genetically modifying the cell to produce type VII collagen.

29. A cell produced by the method of claim 27.

30. A cell produced by the method of claim 28.

31. A cell obtained from a blister of a patient with dystrophic epidermolysis bullosa that is genetically modified to produce type VII collagen.

32. A plasmid for producing a lentiviral vector, comprising an EF1α promoter, and a COL7A1 gene provided downstream of the EF1α promoter.

33. A lentiviral vector, comprising an EF1α promoter, and a COL7A1 gene provided downstream of the EF1α promoter.


SEQUENCE LISTING (6 sequences)

SEQ ID NO: 1 - DNA, 8835 bp, Homo sapiens
atgacgctgcggcttctggtggccgcgctctgcgccgggatcctggcagaggcgccccgagtgcgagcccagcacagggagagagtgacctgcacgcgcctttacgccgctgacattgtgttcttactggatggctcctcatccattggccgcagcaatttccgcgaggtccgcagctttctcgaagggctggtgctgcctttctctggagcagccagtgcacagggtgtgcgctttgccacagtgcagtacagcgatgatccacggacagagttcggcctggatgcacttggctctgggggtgatgtgatccgcgccatccgtgagcttagctacaaggggggcaacactcgcacaggggctgcaattctccatgtggctgaccatgtcttcctgccccagctggcccgacctggtgtccccaaggtctgcatcctgatcacagacgggaagtcccaggacctggtggacacagctgcccaaaggctgaaggggcagggggtcaagctatttgctgtggggatcaagaatgctgaccctgaggagctgaagcgagttgcctcacagcccaccagtgacttcttcttcttcgtcaatgacttcagcatcttgaggacactactgcccctcgtttcccggagagtgtgcacgactgctggtggcgtgcctgtgacccgacctccggatgactcgacctctgctccacgagacctggtgctgtctgagccaagcagccaatccttgagagtacagtggacagcggccagtggccctgtgactggctacaaggtccagtacactcctctgacggggctgggacagccactgccgagtgagcggcaggaggtgaacgtcccagctggtgagaccagtgtgcggctgcggggtctccggccactgaccgagtaccaagtgactgtgattgccctctacgccaacagcatcggggaggctgtgagcgggacagctcggaccactgccctagaagggccggaactgaccatccagaataccacagcccacagcctcctggtggcctggcggagtgtgccaggtgccactggctaccgtgtgacatggcgggtcctcagtggtgggcccacacagcagcaggagctgggccctgggcagggttcagtgttgctgcgtgacttggagcctggcacggactatgaggtgaccgtgagcaccctatttggccgcagtgtggggcccgccacttccctgatggctcgcactgacgcttctgttgagcagaccctgcgcccggtcatcctgggccccacatccatcctcctttcctggaacttggtgcctgaggcccgtggctaccggttggaatggcggcgtgagactggcttggagccaccgcagaaggtggtactgccctctgatgtgacccgctaccagttggatgggctgcagccgggcactgagtaccgcctcacactctacactctgctggagggccacgaggtggccacccctgcaaccgtggttcccactggaccagagctgcctgtgagccctgtaacagacctgcaagccaccgagctgcccgggcagcgggtgcgagtgtcctggagcccagtccctggtgccacccagtaccgcatcattgtgcgcagcacccagggggttgagcggaccctggtgcttcctgggagtcagacagcattcgacttggatgacgttcaggctgggcttagctacactgtgcgggtgtctgctcgagtgggtccccgtgagggcagtgccagtgtcctcactgtccgccgggagccggaaactccacttgctgttccagggctgcgggttgtggtgtcagatgcaacgcgagtgagggtggcctggggacccgtccctggagccagtggatttcggattagctggagcacaggcagtggtccggagtccagccagacactgcccccagactctactgccacagacatcacagggctgcagcctggaaccacctaccaggtggctgtgtcggtactgcgaggcagagaggagggccctgctgcagtcatcgtggctcgaacggacccactgggcccagtgaggacggtccatgtgactcaggccagcagctcatctgtcaccattacctggaccagggttcctggcgccacaggatacagggtttcctggcactcagcccacggcccagagaaatcccagttggtttctggggaggccacggtggctgagctggatggactggagccagatactgagtatacggtgcatgtgagggcccatgtggctggcgtggatgggccccctgcctctgtggttgtgaggactgcccctgagcctgtgggtcgtgtgtcgaggctgcagatcctcaatgcttccagcgacgttctacggatcacctgggtaggggtcactggagccacagcttacagactggcctggggccggagtgaaggcggccccatgaggcaccagatactcccaggaaacacagactctgcagagatccggggtctcgaaggtggagtcagctactcagtgcgagtgactgcacttgtcggggaccgcgagggcacacctgtctccattgttgtcactacgccgcctgaggctccgccagccctggggacgcttcacgtggtgcagcgcggggagcactcgctgaggctgcgctgggagccggtgcccagagcgcagggcttccttctgcactggcaacctgagggtggccaggaacagtcccgggtcctggggcccgagctcagcagctatcacctggacgggctggagccagcgacacagtaccgcgtgaggctgagtgtcctagggccagctggagaagggccctctgcagaggtgactgcgcgcactgagtcacctcgtgttccaagcattgaactacgtgtggtggacacctcgatcgactcggtgactttggcctggactccagtgtccagggcatccagctacatcctatcctggcggccactcagaggccctggccaggaagtgcctgggtccccgcagacacttccagggatctcaagctcccagcgggtgacagggctagagcctggcgtctcttacatcttctccctgacgcctgtcctggatggtgtgcggggtcctgaggcatctgtcacacagacgccagtgtgcccccgtggcctggcggatgtggtgttcctaccacatgccactcaagacaatgctcaccgtgcggaggctacgaggagggtcctggagcgtctggtgttggcacttgggcctcttgggccacaggcagttcaggttggcctgctgtcttacagtcatcggccctccccactgttcccactgaatggctcccatgaccttggcattatcttgcaaaggatccgtgacatgccctacatggacccaagtgggaacaacctgggcacagccgtggtcacagctcacagatacatgttggcaccagatgctcctgggcgccgccagcacgtaccaggggtgatggttctgctagtggatgaacccttgagaggtgacatattcagccccatccgtgaggcccaggcttctgggcttaatgtggtgatgttgggaatggctggagcggacccagagcagctgcgtcgcttggcgccgggtatggactctgtccagaccttcttcgccgtggatgatgggccaagcctggaccaggcagtcagtggtctggccacagccctgtgtcaggcatccttcactactcagccccggccagagccctgcccagtgtattgtccaaagggccagaagggggaacctggagagatgggcctgagaggacaagttgggcctcctggcgaccctggcctcccgggcaggaccggtgctcccggcccccaggggccccctggaagtgccactgccaagggcgagaggggcttccctggagcagatgggcgtccaggcagccctggccgcgccgggaatcctgggacccctggagcccctggcctaaagggctctccagggttgcctggccctcgtggggacccgggagagcgaggacctcgaggcccaaagggggagccgggggctcccggacaagtcatcggaggtgaaggacctgggcttcctgggcggaaaggggaccctggaccatcgggcccccctggacctcgtggaccactgggggacccaggaccccgtggccccccagggcttcctggaacagccatgaagggtgacaaaggcgatcgtggggagcggggtccccctggaccaggtgaaggtggcattgctcctggggagcctgggctgccgggtcttcccggaagccctggaccccaaggccccgttggcccccctggaaagaaaggagaaaaaggtgactctgaggatggagctccaggcctcccaggacaacctgggtctccgggtgagcagggcccacggggacctcctggagctattggccccaaaggtgaccggggctttccagggcccctgggtgaggctggagagaagggcgaacgtggacccccaggcccagcgggatcccgggggctgccaggggttgctggacgtcctggagccaagggtcctgaagggccaccaggacccactggccgccaaggagagaagggggagcctggtcgccctggggaccctgcagtggtgggacctgctgttgctggacccaaaggagaaaagggagatgtggggcccgctgggcccagaggagctaccggagtccaaggggaacggggcccacccggcttggttcttcctggagaccctggccccaagggagaccctggagaccggggtcccattggccttactggcagagcaggacccccaggtgactcagggcctcctggagagaagggagaccctgggcggcctggccccccaggacctgttggcccccgaggacgagatggtgaagttggagagaaaggtgacgagggtcctccgggtgacccgggtttgcctggaaaagcaggcgagcgtggccttcggggggcacctggagttcgggggcctgtgggtgaaaagggagaccagggagatcctggagaggatggacgaaatggcagccctggatcatctggacccaagggtgaccgtggggagccgggtcccccaggacccccgggacggctggtagacacaggacctggagccagagagaagggagagcctggggaccgcggacaagagggtcctcgagggcccaagggtgatcctggcctccctggagcccctggggaaaggggcattgaagggtttcggggacccccaggcccacagggggacccaggtgtccgaggcccagcaggagaaaagggtgaccggggtccccctgggctggatggccggagcggactggatgggaaaccaggagccgctgggccctctgggccgaatggtgctgcaggcaaagctggggacccagggagagacgggcttccaggcctccgtggagaacagggcctccctggcccctctggtccccctggattaccgggaaagccaggcgaggatggcaaacctggcctgaatggaaaaaacggagaacctggggaccctggagaagacgggaggaagggagagaaaggagattcaggcgcctctgggagagaaggtcgtgatggccccaagggtgagcgtggagctcctggtatccttggaccccaggggcctccaggcctcccagggccagtgggccctcctggccagggttttcctggtgtcccaggaggcacgggccccaagggtgaccgtggggagactggatccaaaggggagcagggcctccctggagagcgtggcctgcgaggagagcctggaagtgtgccgaatgtggatcggttgctggaaactgctggcatcaaggcatctgccctgcgggagatcgtggagacctgggatgagagctctggtagcttcctgcctgtgcccgaacggcgtcgaggccccaagggggactcaggcgaacagggccccccaggcaaggagggccccatcggctttcctggagaacgcgggctgaagggcgaccgtggagaccctggccctcaggggccacctggtctggcccttggggagaggggcccccccgggccttccggccttgccggggagcctggaaagcctggtattcccgggctcccaggcagggctgggggtgtgggagaggcaggaaggccaggagagaggggagaacggggagagaaaggagaacgtggagaacagggcagagatggccctcctggactccctggaacccctgggccccccggaccccctggccccaaggtgtctgtggatgagccaggtcctggactctctggagaacagggaccccctggactcaagggtgctaagggggagccgggcagcaatggtgaccaaggtcccaaaggagacaggggtgtgccaggcatcaaaggagaccggggagagcctggaccgaggggtcaggacggcaacccgggtctaccaggagagcgtggtatggctgggcctgaagggaagccgggtctgcagggtccaagaggcccccctggcccagtgggtggtcatggagaccctggaccacctggtgccccgggtcttgctggccctgcaggaccccaaggaccttctggcctgaagggggagcctggagagacaggacctccaggacggggcctgactggacctactggagctgtgggacttcctggaccccccggcccttcaggccttgtgggtccacaggggtctccaggtttgcctggacaagtgggggagacagggaagccgggagccccaggtcgagatggtgccagtggaaaagatggagacagagggagccctggtgtgccagggtcaccaggtctgcctggccctgtcggacctaaaggagaacctggccccacgggggcccctggacaggctgtggtcgggctccctggagcaaagggagagaagggagcccctggaggccttgctggagacctggtgggtgagccgggagccaaaggtgaccgaggactgccagggccgcgaggcgagaagggtgaagctggccgtgcaggggagcccggagaccctggggaagatggtcagaaaggggctccaggacccaaaggtttcaagggtgacccaggagtcggggtcccgggctcccctgggcctcctggccctccaggtgtgaagggagatctgggcctccctggcctgcccggtgctcctggtgttgttgggttcccgggtcagacaggccctcgaggagagatgggtcagccaggccctagtggagagcggggtctggcaggccccccagggagagaaggaatcccaggacccctggggccacctggaccaccggggtcagtgggaccacctggggcctctggactcaaaggagacaagggagaccctggagtagggctgcctgggccccgaggcgagcgtggggagccaggcatccggggtgaagatggccgccccggccaggagggaccccgaggactcacggggccccctggcagcaggggagagcgtggggagaagggtgatgttgggagtgcaggactaaagggtgacaagggagactcagctgtgatcctggggcctccaggcccacggggtgccaagggggacatgggtgaacgagggcctcggggcttggatggtgacaaaggacctcggggagacaatggggaccctggtgacaagggcagcaagggagagcctggtgacaagggctcagccgggttgccaggactgcgtggactcctgggaccccagggtcaacctggtgcagcagggatccctggtgacccgggatccccaggaaaggatggagtgcctggtatccgaggagaaaaaggagatgttggcttcatgggtccccggggcctcaagggtgaacggggagtgaagggagcctgtggccttgatggagagaagggagacaagggagaagctggtcccccaggccgccccgggctggcaggacacaaaggagagatgggggagcctggtgtgccgggccagtcgggggcccctggcaaggagggcctgatcggtcccaagggtgaccgaggctttgacgggcagccaggccccaagggtgaccagggcgagaaaggggagcggggaaccccaggaattgggggcttcccaggccccagtggaaatgatggctctgctggtcccccagggccacctggcagtgttggtcccagaggccccgaaggacttcagggccagaagggtgagcgaggtccccccggagagagagtggtgggggctcctggggtccctggagctcctggcgagagaggggagcaggggcggccagggcctgccggtcctcgaggcgagaagggagaagctgcactgacggaggatgacatccggggctttgtgcgccaagagatgagtcagcactgtgcctgccagggccagttcatcgcatctggatcacgacccctccctagttatgctgcagacactgccggctcccagctccatgctgtgcctgtgctccgcgtctctcatgcagaggaggaagagcgggtaccccctgaggatgatgagtactctgaatactccgagtattctgtggaggagtaccaggaccctgaagctccttgggatagtgatgacccctgttccctgccactggatgagggctcctgcactgcctacaccctgcgctggtaccatcgggctgtgacaggcagcacagaggcctgtcacccttttgtctatggtggctgtggagggaatgccaaccgttttgggacccgtgaggcctgcgagcgccgctgcccaccccgggtggtccagagccaggggacaggtactgcccaggactga

SEQ ID NO: 2 - protein, 2944 aa, Homo sapiens
MTLRLLVAALCAGILAEAPRVRAQHRERVTCTRLYAADIVFLLDGSSSIGRSNFREVRSFLEGLVLPFSGAASAQGVRFATVQYSDDPRTEFGLDALGSGGDVIRAIRELSYKGGNTRTGAAILHVADHVFLPQLARPGVPKVCILITDGKSQDLVDTAAQRLKGQGVKLFAVGIKNADPEELKRVASQPTSDFFFFVNDFSILRTLLPLVSRRVCTTAGGVPVTRPPDDSTSAPRDLVLSEPSSQSLRVQWTAASGPVTGYKVQYTPLTGLGQPLPSERQEVNVPAGETSVRLRGLRPLTEYQVTVIALYANSIGEAVSGTARTTALEGPELTIQNTTAHSLLVAWRSVPGATGYRVTWRVLSGGPTQQQELGPGQGSVLLRDLEPGTDYEVTVSTLFGRSVGPATSLMARTDASVEQTLRPVILGPTSILLSWNLVPEARGYRLEWRRETGLEPPQKVVLPSDVTRYQLDGLQPGTEYRLTLYTLLEGHEVATPATVVPTGPELPVSPVTDLQATELPGQRVRVSWSPVPGATQYRIIVRSTQGVERTLVLPGSQTAFDLDDVQAGLSYTVRVSARVGPREGSASVLTVRREPETPLAVPGLRVVVSDATRVRVAWGPVPGASGFRISWSTGSGPESSQTLPPDSTATDITGLQPGTTYQVAVSVLRGREEGPAAVIVARTDPLGPVRTVHVTQASSSSVTITWTRVPGATGYRVSWHSAHGPEKSQLVSGEATVAELDGLEPDTEYTVHVRAHVAGVDGPPASVVVRTAPEPVGRVSRLQILNASSDVLRITWVGVTGATAYRLAWGRSEGGPMRHQILPGNTDSAEIRGLEGGVSYSVRVTALVGDREGTPVSIVVTTPPEAPPALGTLHVVQRGEHSLRLRWEPVPRAQGFLLHWQPEGGQEQSRVLGPELSSYHLDGLEPATQYRVRLSVLGPAGEGPSAEVTARTESPRVPSIELRVVDTSIDSVTLAWTPVSRASSYILSWRPLRGPGQEVPGSPQTLPGISSSQRVTGLEPGVSYIFSLTPVLDGVRGPEASVTQTPVCPRGLADVVFLPHATQDNAHRAEATRRVLERLVLALGPLGPQAVQVGLLSYSHRPSPLFPLNGSHDLGIILQRIRDMPYMDPSGNNLGTAVVTAHRYMLAPDAPGRRQHVPGVMVLLVDEPLRGDIFSPIREAQASGLNVVMLGMAGADPEQLRRLAPGMDSVQTFFAVDDGPSLDQAVSGLATALCQASFTTQPRPEPCPVYCPKGQKGEPGEMGLRGQVGPPGDPGLPGRTGAPGPQGPPGSATAKGERGFPGADGRPGSPGRAGNPGTPGAPGLKGSPGLPGPRGDPGERGPRGPKGEPGAPGQVIGGEGPGLPGRKGDPGPSGPPGPRGPLGDPGPRGPPGLPGTAMKGDKGDRGERGPPGPGEGGIAPGEPGLPGLPGSPGPQGPVGPPGKKGEKGDSEDGAPGLPGQPGSPGEQGPRGPPGAIGPKGDRGFPGPLGEAGEKGERGPPGPAGSRGLPGVAGRPGAKGPEGPPGPTGRQGEKGEPGRPGDPAVVGPAVAGPKGEKGDVGPAGPRGATGVQGERGPPGLVLPGDPGPKGDPGDRGPIGLTGRAGPPGDSGPPGEKGDPGRPGPPGPVGPRGRDGEVGEKGDEGPPGDPGLPGKAGERGLRGAPGVRGPVGEKGDQGDPGEDGRNGSPGSSGPKGDRGEPGPPGPPGRLVDTGPGAREKGEPGDRGQEGPRGPKGDPGLPGAPGERGIEGFRGPPGPQGDPGVRGPAGEKGDRGPPGLDGRSGLDGKPGAAGPSGPNGAAGKAGDPGRDGLPGLRGEQGLPGPSGPPGLPGKPGEDGKPGLNGKNGEPGDPGEDGRKGEKGDSGASGREGRDGPKGERGAPGILGPQGPPGLPGPVGPPGQGFPGVPGGTGPKGDRGETGSKGEQGLPGERGLRGEPGSVPNVDRLLETAGIKASALREIVETWDESSGSFLPVPERRRGPKGDSGEQGPPGKEGPIGFPGERGLKGDRGDPGPQGPPGLALGERGPPGPSGLAGEPGKPGIPGLPGRAGGVGEAGRPGERGERGEKGERGEQGRDGPPGLPGTPGPPGPPGPKVSVDEPGPGLSGEQGPPGLKGAKGEPGSNGDQGPKGDRGVPGIKGDRGEPGPRGQDGNPGLPGERGMAGPEGKPGLQGPRGPPGPVGGHGDPGPPGAPGLAGPAGPQGPSGLKGEPGETGPPGRGLTGPTGAVGLPGPPGPSGLVGPQGSPGLPGQVGETGKPGAPGRDGASGKDGDRGSPGVPGSPGLPGPVGPKGEPGPTGAPGQAVVGLPGAKGEKGAPGGLAGDLVGEPGAKGDRGLPGPRGEKGEAGRAGEPGDPGEDGQKGAPGPKGFKGDPGVGVPGSPGPPGPPGVKGDLGLPGLPGAPGVVGFPGQTGPRGEMGQPGPSGERGLAGPPGREGIPGPLGPPGPPGSVGPPGASGLKGDKGDPGVGLPGPRGERGEPGIRGEDGRPGQEGPRGLTGPPGSRGERGEKGDVGSAGLKGDKGDSAVILGPPGPRGAKGDMGERGPRGLDGDKGPRGDNGDPGDKGSKGEPGDKGSAGLPGLRGLLGPQGQPGAAGIPGDPGSPGKDGVPGIRGEKGDVGFMGPRGLKGERGVKGACGLDGEKGDKGEAGPPGRPGLAGHKGEMGEPGVPGQSGAPGKEGLIGPKGDRGFDGQPGPKGDQGEKGERGTPGIGGFPGPSGNDGSAGPPGPPGSVGPRGPEGLQGQKGERGPPGERVVGAPGVPGAPGERGEQGRPGPAGPRGEKGEAALTEDDIRGFVRQEMSQHCACQGQFIASGSRPLPSYAADTAGSQLHAVPVLRVSHAEEEERVPPEDDEYSEYSEYSVEEYQDPEAPWDSDDPCSLPLDEGSCTAYTLRWYHRAVTGSTEACHPFVYGGCGGNANRFGTREACERRCPPRVVQSQGTGTAQD

SEQ ID NO: 3 - DNA, 20 bp, Homo sapiens
accccacagtggggccacta

SEQ ID NO: 4 - DNA, 20 bp, Homo sapiens
gtcaccaatcctgtccctag

SEQ ID NO: 5 - DNA, 20 bp, Homo sapiens
ggggccactagggacaggat

SEQ ID NO: 6 - DNA, 1188 bp, Artificial Sequence
  EF1a promoter
gtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaa